(19) United States
(12) Patent Application Publication
(10) Pub. No.: US 2018/0064738 A1
(43) Pub. Date: Mar. 8, 2018

(54) METHOD OF TREATING INFECTIONS USING SILOXANE DERIVATIVES

(71) Applicant: KIMMERLING HOLDINGS GROUP, LLC, Marietta, GA (US)

(72) Inventors: Bhupesh Kumar PRUSTY, Giebelstadt (DE); Dharam V. ABLASHI, Powder Springs, GA (US); Kirk KIMMERLING, Kennesaw, GA (US)

(21) Appl. No.: 15/517,060

(22) PCT Filed: Oct. 7, 2015
(86) PCT No.: PCT/US2015/054421

§ 371 Date: Apr. 5, 2017

Related U.S. Application Data
(60) Provisional application No. 62/060,759, filed on Oct. 7, 2014.

(51) Int. Cl. A61K 31/695; A61K 31/80; A61K 45/06; A61K 47/10

(52) U.S. Cl. CPC A61K 31/695; A61K 47/10; A61K 45/06; A61K 31/80

(57) ABSTRACT

Pharmaceutical compositions are disclosed that comprise at least one compound of formula I:

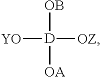

wherein the substituent groups are as defined in the specification, and a pharmaceutically-acceptable excipient. The disclosed pharmaceutical compositions are anti-infective and useful as therapies for the treatment of an infection, including infections associated with a bacteria or a virus. This abstract is intended as a scanning tool for purposes of searching in the particular art and is not intended to be limiting of the present invention.

### CROSS REFERENCE TO RELATED APPLICATIONS

This application claims priority to U.S. application Ser. No. 62/060,759 filed Oct. 7, 2014, the entire disclosure of which is incorporated herein by reference.

### FIELD OF INVENTION

The present invention generally relates to anti-infective compositions and methods of treating an infection in a subject.

### BACKGROUND OF THE INVENTION

Infectious diseases, particularly those caused by viruses, presently result in a significant burden on human populations in terms of economic cost, morbidity and mortality. Many of viral diseases, including chickenpox, influenza, herpes, human immunodeficiency virus (HIV/AIDS), human papillomavirus (HPV), infectious mononucleosis, mumps, measles, rubella, shingles, viral gastroenteritis, viral hepatitis, viral meningitis, and viral pneumonia, can be fatal, particularly in vulnerable populations such as children or the elderly. Of particular concern is that the risk of various viral infections to humans rapidly spreading and potentially becoming pandemics has significantly increased in the last fifty years, in large part due to phenomenal increase in air travel. For example, in 2009, there were over 2.5 billion air travelers worldwide, with nearly 1 billion of those as international travelers crossing national borders. These concerns were borne out in 2009 with the rapid spread of a new strain of H1 N1 influenza virus creating a pandemic.

Viruses are minute microorganisms having no cell structure, and they are broadly classified as DNA viruses or RNA viruses. In some sense, viruses are not living organisms in their own right since they completely depend upon host cells for all aspects that characterize living cells. For example, viruses require host cells for protein synthesis and energy production mechanisms, and viruses completely lack their own metabolic pathways. In short, viruses cannot exist without the cellular machinery of a host cell. Thus, viral infection presents a particularly difficult therapeutic challenge, in part due to the significant difficulty of designing therapeutic agents that attack the various microorganisms without significant collateral damage to the host cells and other cells in the body.

Despite advances in the understanding of the biology of viruses, there is still a scarcity of compounds that are both potent, efficacious, and selective therapeutic agents for the treatment of viral diseases. These needs and other needs are satisfied by the present invention.

### SUMMARY OF INVENTION

In accordance with the purpose(s) of the invention, as embodied and broadly described herein, the invention, in one aspect, relates to pharmaceutical compositions for treating an infection and methods of using such pharmaceutical compositions. For example, the disclosed pharmaceutical compositions can be useful in the treatment of infections associated with a microbe, including, for example, bacteria and viruses.

Disclosed are pharmaceutical compositions, comprising at least one compound of formula I:

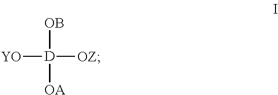

wherein:

D is independently Si, Ti, Al, or Zr;

A, B, Y, and Z are each independently selected from the group consisting of H, (C₁-C₈)alkyl, trifluoro-substituted (C₁-C₈)alkyl, and

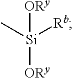

R^{b} is independently

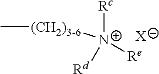

wherein:
R^{c} is (C₁-C₂)alkyl;
R^{d} is (C₁-C₂)alkyl or phenyl;
R^{e} is (C₆-C₂₂)alkyl;
X⁻ is an anion selected from the group consisting of chloride, bromide, fluoride, iodide, sulfonate, and acetate;

each R^{y} is, independently, H, (C₁-C₈)alkyl, or trifluoro-substituted (C₁-C₈)alkyl; and

wherein at least one of A, B, Y, and Z is

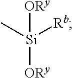

and

a pharmaceutically-acceptable excipient.

Also disclosed are methods of treating an infection in a subject, comprising administering to the mammal an effective amount of a disclosed pharmaceutical composition.

Also disclosed are methods of reducing pain associated with a cutaneous or mucosal membrane lesion caused by a herpes viral infection in a mammal, comprising administering to the mammal an effective amount of a disclosed pharmaceutical composition.

Also disclosed are methods of hastening healing of a cutaneous or mucosal membrane lesion caused by a herpes viral infection in a mammal, comprising administering to the mammal an effective amount of a disclosed pharmaceutical composition.

Also disclosed are kits comprising a disclosed pharmaceutical composition, and at least one of:
a) at least one therapeutic agent known to treat a viral infection;
b) at least one therapeutic agent known to treat a bacterial infection;
c) instructions for treating a viral infection;
d) instructions for treating a bacterial infection;
e) instructions for administering the pharmaceutical composition in connection with treating a viral infection; or
f) instructions for administering the pharmaceutical composition in connection with treating a bacterial infection.

Also disclosed are uses of a disclosed pharmaceutical composition in the manufacture of a medicament for the treatment of an infection.

While aspects of the present invention may be described and claimed in a particular statutory class, such as the system statutory class, this is for convenience only and one of skill in the art will understand that each aspect of the present invention may be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention. In the drawings:

FIG. 1 shows chemical structure of the molecule referred to herein as K-21. The molecular and formula weights for K-21 are also shown on the figure.

FIG. 2 shows representative data for the 50% cytotoxic concentration (CC₅₀) of a representative disclosed compound, K-21, in Vero cells.

FIG. 3 shows representative data for inhibition of HSV-1 infection in Vero cells by a representative disclosed compound, K-21.

FIG. 4 shows representative data for effect of K-21 on induced cytopathy in uninfected Vero cells.

FIG. 5 shows representative data for effect of K-21 on induced cytopathy in infected Vero cells.

FIG. 6 shows representative data for induction of cell death in uninfected (“−HSV-1”) and infected (+HSV-1) Vero cells treated with a representative disclosed compound, K-21.

FIG. 7 shows representative Western blot data for the effect of a representative disclosed compound, K-21, on the expression of the indicated proteins in Vero cells following infection by HSV-1.

FIG. 8 shows the results of a cell viability assay in primary human foreskin fibroblasts to determine cytotoxic dose of K-21.

FIG. 9 shows data from HSV-1 infection in primary human foreskin fibroblasts. Panel (A) shows the cells imaged using an epifluorescence microscope. Panel (B) shows total genomic DNA in infected cells quantified by qPCR. Panel (C) shows plaque assays to check for infectious progeny. Panel (D) shows immunoblotting from total lysate from 24 hour infected cells. Panel (E) shows the effect of K-21 on HSV-1 entry and attachment was studied by qPCR.

FIG. 10 shows representative data from flow cytometry for Vero cells with and without infection with HSV-1 in the presence of K-21 at different dilutions.

FIG. 11 shows data from HHV-6 and HHV-7 infection. Panel (A) shows the cells imaged using an epifluorescence microscope for HSV-6A. Panel (B) shows total genomic DNA in infected cells quantified by qPCR. Panel (C) shows the effect of K-21 on HHV-6A entry and attachment was studied by qPCR. Panel (D) shows immunoblotting from total lysate from 24 hour infected cells for HHV-6. Panel (E) shows shows total genomic DNA in infected cells quantified by qPCR for HHV-7. Panel (F) shows immunoblotting from total lysate from 24 hour infected cells for HHV-7.

FIG. 12 shows immunoblotting for the the effect of K-21 on HSV-1 replication and growth.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION OF THE INVENTION

The present invention may be understood more readily by reference to the following detailed description of the invention and the Examples included therein.

As employed above and throughout the disclosure, the following terms, unless otherwise indicated, shall be understood to have the following meanings.

“Alkyl,” as used herein, refers to an optionally-substituted, saturated straight, branched, or cyclic hydrocarbon having from about 1 to about 20 carbon atoms (and all combinations and subcombinations of ranges and specific numbers of carbon atoms therein), with from about 1 to about 8 carbon atoms or 1 to 6 carbon atoms (C₁-C₆) being preferred, and with from about 1 to about 4 carbon atoms. Alkyl groups include, but are not limited to, methyl, ethyl, n-propyl, isopropyl, n-butyl, isobutyl, t-butyl, n-pentyl, cyclopentyl, cyclopropyl, isopentyl, neopentyl, n-hexyl, isohexyl, cyclohexyl, cyclooctyl, adamantyl, 3-methylpentyl, 2,2-dimethylbutyl, and 2,3-dimethylbutyl. A branched alkyl group has at least 3 carbon atoms (e.g., an isopropyl group), and in various embodiments, has up to 6 carbon atoms, i.e., a branched lower alkyl group. A branched alkyl group has at least 3 carbon atoms (e.g., an isopropyl group), and in various embodiments, has up to 6 carbon atoms, i.e., a branched lower alkyl group.

“Alkenyl,” as used herein, refers to an optionally-substituted, singly unsaturated, straight, branched, or cyclic hydrocarbon having from about 2 to about 20 carbon atoms (and all combinations and subcombinations of ranges and specific numbers of carbon atoms therein), with from about 2 to about 8 carbon atoms or 2 to 6 carbon atoms (C₂-C₆) being preferred. Alkenyl groups include, but are not limited to, ethenyl (or vinyl), allyl, propenyl, butenyl, pentenyl, cyclopentenyl, hexenyl, and octenyl.

“Alkylenyl,” as used herein, refer to the subsets of alkyl groups, as defined herein, including the same residues as alkyl but having two points of attachment within a chemical structure. Examples of (C₁-C₆)alkylenyl include methylenyl (—CH₂—), ethylenyl (—CH₂CH₂—), propylenyl (—CH₂CH₂CH₂—), and dimethylpropylenyl (—CH₂C(CH₃)₂CH₂—).

“Aryl,” as used herein, refers to an optionally-substituted, mono-, di-, tri-, or other multicyclic aromatic ring system having from about 5 to about 50 carbon atoms (and all combinations and subcombinations of ranges and specific numbers of carbon atoms therein), with from about 6 to about 10 carbons (C₆-C₁₀) being preferred. Non-limiting examples include, for example, phenyl, naphthyl, anthracenyl, and phenanthrenyl.

As used herein, the terms “optionally-substituted” or “substituted” are intended to refer to the optional replacement of up to four hydrogen atoms with up to four independently selected substituent groups as defined herein. Unless otherwise specified, suitable substituent groups independently include hydroxyl, nitro, amino, imino, cyano, halo, thio, sulfonyl, aminocarbonyl, carbonylamino, carbonyl, oxo, guanidine, carboxyl, formyl, alkyl, perfluoroalkyl, alkylamino, dialkylamino, alkoxy, alkoxyalkyl, alkylcarbonyl, arylcarbonyl, alkylthio, aryl, heteroaryl, a heterocyclic ring, cycloalkyl, hydroxyalkyl, carboxyalkyl, haloalkyl, alkenyl, alkynyl, arylalkyl, aryloxy, heteroaryloxy, heteroarylalkyl, and the like. Substituent groups that have one or more available hydrogen atoms can in turn optionally bear further independently selected substituents, to a maximum of three levels of substitutions. For example, the term “optionally-substituted alkyl” is intended to mean an alkyl group that can optionally have up to four of its hydrogen atoms replaced with substituent groups as defined above (i.e., a first level of substitution), wherein each of the substituent groups attached to the alkyl group can optionally have up to four of its hydrogen atoms replaced by substituent groups as defined above (i.e., a second level of substitution), and each of the substituent groups of the second level of substitution can optionally have up to four of its hydrogen atoms replaced by substituent groups as defined above (i.e., a third level of substitution).

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” In this manner, slight variations from a stated value can be used to achieve substantially the same results as the stated value. Also, the disclosure of ranges is intended as a continuous range including every value between the minimum and maximum values recited as well as any ranges that can be formed by such values. Also disclosed herein are any and all ratios (and ranges of any such ratios) that can be formed by dividing a recited numeric value into any other recited numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present invention.

As used herein, the phrase “substantially” means have no more than about 10% difference between the target and actual level, preferably less than about 5% difference, more preferably, less than about 1% difference.

The term “viral infection” or “infection associated with a virus” refers to the introduction of a virus into cells or tissues, e.g., an influenza virus. In general, the introduction of a virus is also associated with replication of the virus. Viral infection may be determined by measuring virus antibody titer in samples of a biological fluid, such as blood, using, e.g., enzyme immunoassay. Other suitable diagnostic methods include molecular based techniques, such as RT-PCR, direct hybrid capture assay, nucleic acid sequence based amplification, and the like. A virus may infect a particular organ, e.g., lung, and cause disease, e.g., with localized effects (such as respiratory impairment and edema) and systemic effects.

The term “bacterial infection” or “infection associated with bacteria” refers to the introduction of bacteria, e.g., Staphylococcus aureus bacteria, into the body or exposure of a cell to bacteria. In general, the introduction of bacteria is also associated with replication of the bacteria. Bacterial infection may be determined by measuring bacterial antibody titer in samples of a biological fluid, such as blood, using, e.g., enzyme immunoassay. Other suitable diagnostic methods include molecular based techniques, such as RT-PCR, direct hybrid capture assay, nucleic acid sequence based amplification, and the like. Bacteria may infect an particular organ, e.g., lung, and cause disease, e.g., with localized effects (such as respiratory impairment and edema) and systemic effects.

The term “fungal infection” or “infection associated with fungus” refers to the introduction of fungus, e.g., Candida albicans fungus, into the body or exposure of a cell to a fungus. In general, the introduction of a fungus is also associated with replication of the fungus. A fungal infection may be determined by measuring fungal antibody titer in samples of a biological fluid, such as blood, using, e.g., enzyme immunoassay. Other suitable diagnostic methods include molecular based techniques, such as RT-PCR, direct hybrid capture assay, nucleic acid sequence based amplification, and the like. A fungus may infect an particular organ, e.g., lung, and cause disease, e.g., with localized effects (such as respiratory impairment and edema) and systemic effects.

The term “protozoan infection” or “infection associated with a protozoan” refers to the introduction of a protozoan, e.g., Plasmodium falciparum, into the body or exposure of a cell to a protozoan. In general, the introduction of a protozoan is also associated with replication of the protozoan. A protozoan infection may be determined by measuring protozoan antibody titer in samples of a biological fluid, such as blood, using, e.g., enzyme immunoassay. Other suitable diagnostic methods include molecular based techniques, such as RT-PCR, direct hybrid capture assay, nucleic acid sequence based amplification, and the like. A fungus may infect an particular organ, e.g., lung, and cause disease, e.g., with localized effects (such as respiratory impairment and edema) and systemic effects.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder, e.g. an infection with a virus or bacteria. The term “patient” includes human and veterinary subjects. In some aspects of the disclosed methods, the subject has been diagnosed with a need for treatment of at least one infection prior to the administering step.

As used herein, the term “treatment” or “treating” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; supportive treatment, that is, treatment employed to supplement another specific therapy directed toward the improvement of the associated disease, pathological condition, or disorder; and prophylactic treatment, that is, treatment directed to preventing a disease or disorder in a subject, preventing the occurrence of symptoms in a subject with a disease or disorder, preventing the recurrence of symptoms in a subject with a disease or disorder, and/or decreasing the severity of frequency of outward symptoms of disease or disorder in a subject. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease.

As used herein, the term “prophylaxis” or “prophylactic” refers to the complete prevention of infection, the prevention of occurrence of symptoms in an infected subject, the prevention of recurrence of symptoms in an infected subject, or a decrease in severity or frequency of outward symptoms of infection or disease in the subject.

As used herein, the term “prevent” or “preventing” refers to precluding, averting, obviating, forestalling, stopping, or hindering something from happening, especially by advance action. It is understood that where the terms “reduce,” “inhibit” or “prevent” are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the phrase “identified to be in need of treatment for a disorder,” or the like, refers to selection of a subject based upon need for treatment of the disorder. For example, a subject can be identified as having a need for treatment of a disorder based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The terms “co-administer(s)”, “co-administering”, and “co-administration” all refer to with respect to compounds or compositions, is meant either simultaneous administration or any manner of separate sequential administration of at least one disclosed compound, with at least one pharmaceutically active agent, such as, but not limited to, those agents included in antimicrobial therapy. Preferably, if the administration is not simultaneous, the compounds are administered in a close time proximity to each other. Furthermore, it does not matter if the compounds are administered in the same dosage form, e.g. one compound may be administered topically and another compound may be administered orally. “Substantially simultaneously” means that the compound, i.e. a disclosed compound, is typically administered during or within a reasonably short time either before or after the administration of other compounds, such as a pharmaceutically active agent that treats the disease in question. Additionally, “co-administration”, “co-administer(s)”, and “co-administering” include administering more than one dose of the pharmaceutically active agent within 24 hours of a dose of a disclosed compound. In other words, the disclosed compound need not be administered again before or with every administration of a pharmaceutically active agent, but may be administered intermittently during the course of treatment. “Co-administration”, “co-administer(s)”, and “co-administering” also includes administering a pharmaceutically active agent and a disclosed compound as a part of one or more pharmaceutical compositions, and such one or more pharmaceutical compositions may contain a co-formulation of a disclosed compound and a pharmaceutically active agent or individual formulations of a pharmaceutically active agent and a disclosed compound.

It is understood that co-administration of a disclosed compound and an anti-microbial agent or other therapeutic agent can be independently co-administered by any appropriate route of administration. The active agents, i.e. a disclosed compound and an anti-microbial agent or other therapeutic agent, can be administered by the same or different routes of administration, as appropriate. For example, one of the active ingredients can be administered orally and the other administered orally or by some other appropriate route of administration. Alternatively, the combination of active ingredients can be concurrently orally administered. In a further example, consistent with this understanding, one of the active ingredients can be administered parenterally, for example, intravenously, intramuscularly, subcutaneously, topically, intravaginally, rectally, intranasally, inhaled, intrathecally, intraocularly, and at least one other active ingredient administrated by a similar or distinct route of administration. Moreover, it is understood that a disclosed compound and an anti-viral agent or other therapeutic agent can be co-administered or independently administered by distinct routes of administration, such as parenterally, orally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, or intrathecally.

As used herein, “combination therapy” (or “co-therapy”) refers to the administration of a disclosed compound and an anti-microbial agent or other therapeutic agent during the course of therapy or treatment for an infection. Such combination therapy may involve the administration of the disclosed compound before, during, and/or after the administration of the anti-microbial agent or other therapeutic agent administered to ameliorate, treat, reverse, or cure the infection or symptoms associated with the infection. The administration of the disclosed compound may be separated in time from the administration of anti-microbial agent or other therapeutic agent by up to several weeks, and may precede it or follow it, but more commonly the administration of the disclosed compound will accompany at least one aspect of the administration of the anti-microbial agent or other therapeutic agent.

As used herein, “concurrently” means (1) simultaneously in time, or (2) at different times during the course of a common treatment schedule.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount or dosage that can effectively prevent a disease or disorder in a subject, prevent the occurrence of symptoms in a subject with a disease or disorder, prevent the recurrence of symptoms in a subject with a disease or disorder, and/or decrease the severity of frequency of outward symptoms of a disease or disorder in a subject.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro- drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

The term “excipient,” as used herein, refers to a compound that is used to prepare a pharmaceutical composition, and is generally safe, non-toxic and neither biologically nor otherwise undesirable, and includes excipients that are acceptable for veterinary use as well as human pharmaceutical use. The compounds of this invention may be administered alone but will generally be administered in admixture with one or more suitable pharmaceutical excipients, diluents or carriers selected with regard to the intended route of administration and standard pharmaceutical practice.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions that can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

In one aspect, the invention relates to relates to pharmaceutical compositions for treating an infection. Accordingly, in various aspects, the invention is directed to pharmaceutical compositions, comprising:

at least one compound of formula I:

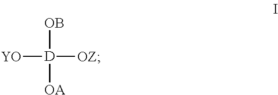

wherein:

D is independently Si, Ti, Al, or Zr;

A, B, Y, and Z are each independently selected from the group consisting of H, (C₁-C₈)alkyl, trifluoro-substituted (C₁-C₈)alkyl, and

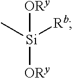

R^{b} is independently

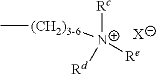

wherein:
R^{c} is (C₁-C₂)alkyl;
R^{d} is (C₁-C₂)alkyl or phenyl;
R^{e} is (C₆-C₂₂)alkyl;
X⁻ is an anion selected from the group consisting of chloride, bromide, fluoride, iodide, sulfonate, and acetate;

each R^{y} is, independently, H, (C₁-C₈)alkyl, or trifluoro-substituted (C₁-C₈)alkyl; and

wherein at least one of A, B, Y, and Z is

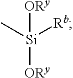

and

a pharmaceutically-acceptable excipient.

In a further aspect of the pharmaceutical composition comprising the compound of formula I, each of A, B, Y, and Z is

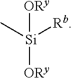

In a further aspect of the pharmaceutical composition comprising the compound of formula I, D is Si.

In a further aspect of the pharmaceutical composition comprising the compound of formula I, R^{c} is (C₁)alkyl. In a still further aspect of the pharmaceutical composition comprising the compound of formula I, R^{d} is (C₁)alkyl. In a yet further aspect of the pharmaceutical composition comprising the compound of formula I, R^{e} is (C₁₈)alkyl.

In a further aspect of the pharmaceutical composition comprising the compound of formula I, X is chloride.

In a further aspect of the pharmaceutical composition comprising the compound of formula I, R^{b} is independently —(C₃-C₆ alkylenyl)—(dimethyl)—(C₆-C₂₂ alkyl) quaternary ammonium chloride or —(C₃-C₆ alkylenyl)—(methyl)—(phenyl)—(C₆-C₂₂ alkyl) quaternary ammonium chloride. In a still further aspect of the pharmaceutical composition comprising the compound of formula I, R^{b} is —(C₃ alkylenyl)—(dimethyl)—(C₁₈ alkyl) quaternary ammonium chloride.

In a further aspect of the pharmaceutical composition comprising the compound of formula I, each R^{y} is H.

In a further aspect of the pharmaceutical composition comprising the compound of formula I, the compound of formula I has the formula:

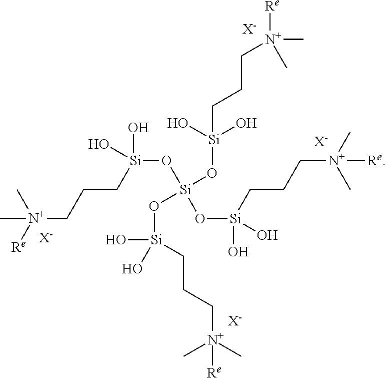

In a further aspect of the pharmaceutical composition comprising the compound of formula I, the compound of formula I has the formula:

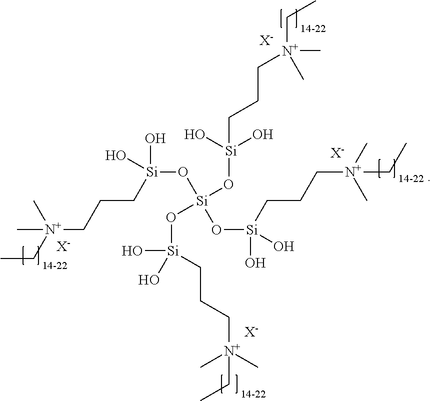

In a further aspect of the pharmaceutical composition comprising the compound of formula I, the compound of formula I has the formula:

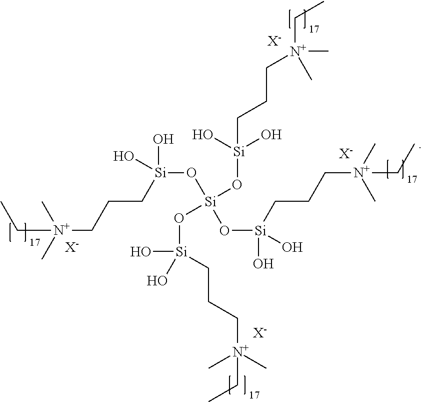

In a further aspect of the pharmaceutical composition comprising the compound of formula I, the compound of formula I has the formula:

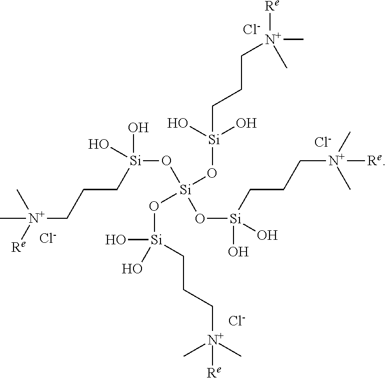

In a further aspect of the pharmaceutical composition comprising the compound of formula I, the compound of formula I has the formula:

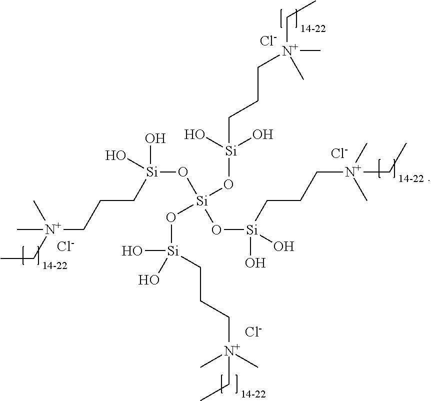

In a further aspect of the pharmaceutical composition comprising the compound of formula I, the compound of formula I has the formula:

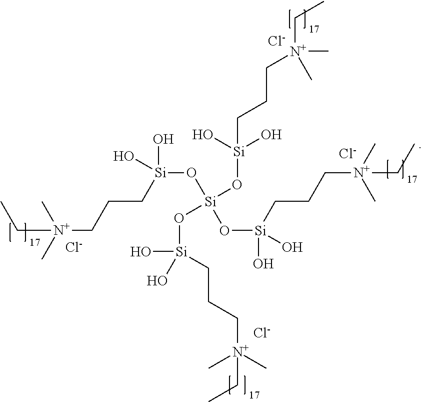

In a further aspect, the pharmaceutical composition comprising the compound of formula I, further comprises at least one byproduct of the disclosed methods of making the compound of formula I. In a still further aspect, the pharmaceutical composition comprising the compound of formula I, further comprises at least one byproduct of the disclosed methods of making the compound of formula I, and the byproducts are associated with a hydrolysis and condensation reaction occurring under the normal reaction conditions set forth herein. In a yet further aspect, the pharmaceutical composition comprising the compound of formula I, further comprises 0-3 byproducts of the disclosed methods of making the compound of formula I. In an even further aspect, the pharmaceutical composition comprising the compound of formula I, further comprises 1-3 byproducts of the disclosed methods of making the compound of formula I.

In a further aspect, the pharmaceutical composition comprising the compound of formula I, further comprises a compound of formula II:

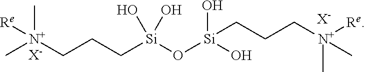

In a further aspect, the pharmaceutical composition comprising the compound of formula I, further comprises a compound of formula II; and the compound of formula II has the formula:

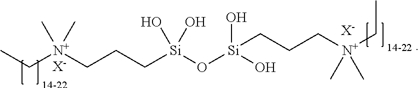

In a further aspect, the pharmaceutical composition comprising the compound of formula I, further comprises a compound of formula II; and the compound of formula II has the formula:

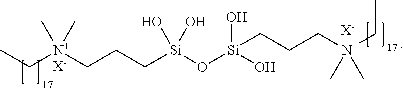

In a further aspect, the pharmaceutical composition comprising the compound of formula I, further comprises a compound of formula II; and the compound of formula II has the formula:

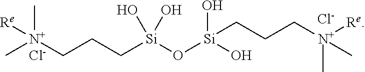

In a further aspect, the pharmaceutical composition comprising the compound of formula I, further comprises a compound of formula II; and the compound of formula II has the formula:

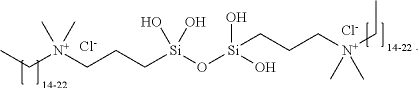

In a further aspect, the pharmaceutical composition comprising the compound of formula I, further comprises a compound of formula II; and the compound of formula II has the formula:

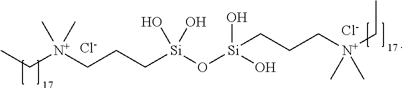

In a further aspect, the pharmaceutical composition comprising the compound of formula I, further comprises a compound of formula III:

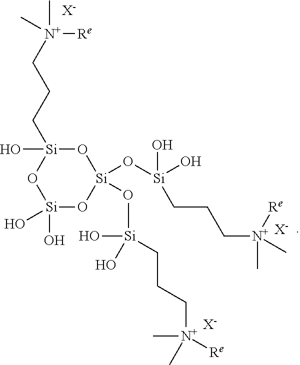

In a further aspect, the pharmaceutical composition comprising the compound of formula I, further comprises a compound of formula III; and the compound of formula III has the formula:

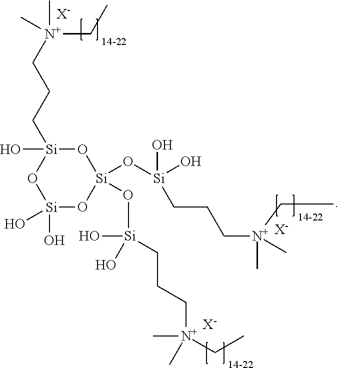

In a further aspect, the pharmaceutical composition comprising the compound of formula I, further comprises a compound of formula III; and the compound of formula III has the formula:

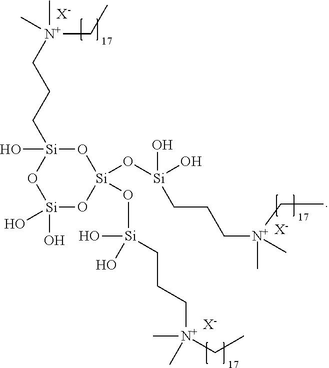

In a further aspect, the pharmaceutical composition comprising the compound of formula I, further comprises a compound of formula III; and the compound of formula III has the formula:

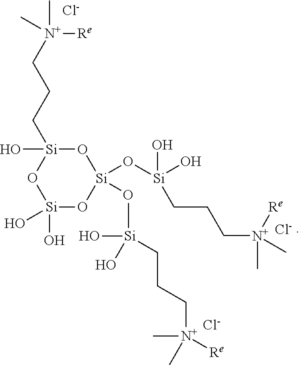

In a further aspect, the pharmaceutical composition comprising the compound of formula I, further comprises a compound of formula III; and the compound of formula III has the formula:

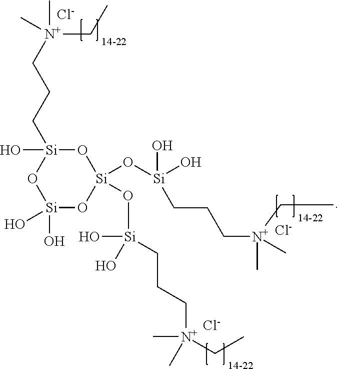

In a further aspect, the pharmaceutical composition comprising the compound of formula I, further comprises a compound of formula III; and the compound of formula III has the formula:

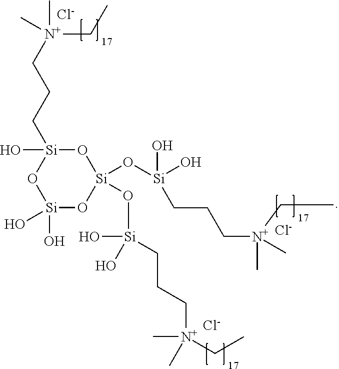

In a further aspect, the pharmaceutical composition comprising the compound of formula I, further comprises at least one therapeutic agent.

In a further aspect, the pharmaceutical composition comprising the compound of formula I, further comprises at least one therapeutic agent; and the at least one therapeutic agent comprises at least one antiviral agent.

In a further aspect of the pharmaceutical composition comprising the compound of formula I, the antiviral agent is a DNA synthesis inhibitor. In a still further aspect, the DNA synthesis inhibitor is a nucleoside analogue. In a yet further aspect, the DNA synthesis inhibitor is idoxuridine, trifluridine, vidarabine, acyclovir, penciclovir, famiciclovir, ganciclovir, cidofovir, valaciclovir, valganciclovir, fosccarnet, or a combination thereof.

In a further aspect of the pharmaceutical composition comprising the compound of formula I, the antiviral agent is an RNA synthesis inhibitor. In a still further aspect, the RNA synthesis inhibitor is a nucleoside analogue.

In a further aspect of the pharmaceutical composition comprising the compound of formula I, the antiviral agent is an HIV antiviral agent. In a still further aspect, the HIV antiviral agent is delavirdine, efavirenz, etravirine, nevirapine, rilpivirine, lersivirine, abacavir, didansine, emtricitabine, lamivudine, stavudine, tenofovir, zidovudine, elvucitabine, atazanavir, darunavir, fosamprenavir, indinavir, lopinavir, nelfinavir, ritonavir, saquinavir, tipranavir, raltegravir, dolutegravir, elvitegravir, enfuvirtide, maraviroc, cenicriviroc, ibalizumab, or a combination thereof.

In a further aspect of the pharmaceutical composition comprising the compound of formula I, the antiviral agent is an influenza antiviral agent. In a still further aspect, the influenza antiviral agent is amantadine, rimantadine, oseltamivir, zanamivir, peramivir, laninamivir octanoate, ribavirin, viramidine, 6-fluoro-3-hydroxy-2-pyrazinecarboxamide, 2′-deoxy-2′-fluoroguanosine, pyrazofurin, carbodine, cyclopenenyl cytosine, beraprost, nileprost, iloprost, cicaprost, eptaloprost, ciprosten, or a combination thereof.

In a further aspect, the pharmaceutical composition comprising the compound of formula I, further comprises at least one therapeutic agent; and the at least one therapeutic agent comprises at least one antibacterial agent. In a still further aspect, the antibacterial agent is amikacin, amoxicillin, amoxicillin/clavulanate, aztreonam, azithromycin, cefaclor, cefadroxil, cephalexin, cefazolin, cefixime, cefotaxime, cefotetan, cefoxitin, cefpodoxime, ceftaroline fosamil, ceftazidime, ceftriaxone, cefuroxime, cephalexin, cephradine, chloramphenicol, cilastatin/imipenem, ciprofloxacin, clavulanate/ticarcillin, clarithromycin, clindamycin, clofazimine, colistin, daptomycin, demeclocycline, doripenem, doxycycline, ertapenem, fosfomycin/trometamol, fusidic acid, gentamicin, grepafloxacin, kanamycin, levofloxacin, lincomycin, linezolid, lymecycline, meropenem, metronidazole, minocycline, moxifloxacin, nafcillin, nalidixic acid, netilmicin, nitrofuratoin, norfloxacin, ofloxacin, oxacillin, oxytetracycline, penicillin, phenoxymethylpenicillin, piperacillin, pivmecillinam, polymyxin B, rifaximin, streptomycin, sulfadiazine, sulfamethoxazole/trimethoprim, sulfisoxazole, telithromycin, tetracycline, tobramycin, trimethoprim/sulfamethoxazole, vancomycin, or a combination thereof.

In a further aspect, the pharmaceutical composition comprising the compound of formula I, further comprises at least one therapeutic agent; the at least one therapeutic agent comprises at least one antibacterial agent; and the at least one antibacterial agent is an antituberculosis agent. In a still further aspect, the antituberculosis agent is capreomycin, clofazimine, cycloserine, ethambutol, ethionamide, isoniazid, pyrazinamide, rifabutin, rifampin, rifapentine, or a combination thereof.

In a further aspect, the pharmaceutical composition comprising the compound of formula I, further comprises at least one therapeutic agent; and the at least one therapeutic agent comprises at least one antiprotozoan agent. In a still further aspect, the antiprotozoan agent is eflornithine, furazolidone, iodoquinol, melarsoprol, metronidazole, ornidazole, paromomycin sulfate, pentamidine, pyrimethamine, tinidazole, amodiaquine, arteether, artemisinin, artemether, artesunate, atovaquone, chloroquine, clindamycin, dihydroartemisinin, doxycycline, halofantrine, mefloquine, primaquine, proguanil, pyrimethamine, quinimax, quinidine, proguanil, pyrimethamine, sulfadoxine, sulfamethoxypyridazine, or a combination thereof. In a yet further aspect, the antiprotozoan agent is eflornithine, furazolidone, iodoquinol, melarsoprol, metronidazole, ornidazole, paromomycin sulfate, pentamidine, pyrimethamine, tinidazole, or a combination thereof.

In a further aspect, the pharmaceutical composition comprising the compound of formula I, further comprises at least one therapeutic agent; the at least one therapeutic agent comprises at least one antiprotozoan agent; and the at least one antiprotozoan agent is an antimalarial agent. In a still further aspect, the antimalarial agent is amodiaquine, arteether, artemisinin, artemether, artesunate, atovaquone, chloroquine, clindamycin, dihydroartemisinin, doxycycline, halofantrine, mefloquine, primaquine, proguanil, pyrimethamine, quinimax, quinidine, proguanil, pyrimethamine, sulfadoxine, sulfamethoxypyridazine, or a combination thereof.

In a further aspect, the pharmaceutical composition comprising the compound of formula I, further comprises a solvent. In a still further aspect, the pharmaceutical composition comprising the compound of formula I, further comprises a solvent selected from the group consisting of acetic acid, acetone, anisole, 1,2-butanediol, 1,3-butanediol, 1,4-butanediol, 1-butanol, 2-butanol, dimethyl sulfoxide, ethanol, ethyl acetate, ethyl ether, ethyl formate, formic acid, heptane, isobutyl acetate, isopropyl acetate, methyl acetate, 3-methyl-1-butanol, butyl acetate, methylethyl ketone, tert-butylmethyl ether, methylisobutyl ketone, 2-methyl-1-propanol, pentane, 1-pentanol, 1-propanol, 2-propanol, propyl acetate, or a combination thereof. In a yet further aspect, the pharmaceutical composition comprising the compound of formula I, further comprises a solvent selected from the group consisting of acetic acid, acetone, anisole, 1-butanol, 2-butanol, dimethyl sulfoxide, ethanol, ethyl acetate, ethyl ether, ethyl formate, formic acid, heptane, isobutyl acetate, isopropyl acetate, methyl acetate, 3-methyl-1-butanol, butyl acetate, methylethyl ketone, tert-butylmethyl ether, methylisobutyl ketone, 2-methyl-1-propanol, pentane, 1-pentanol, 1-propanol, 2-propanol, propyl acetate, or a combination thereof.

In various aspects, compounds of formula I:

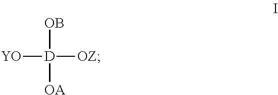

are derived by the hydrolysis of the precursors:

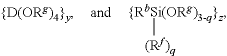

R^{f} is independently is hydroxyl, (C₁-C₈)alkyl, or substituted (C₁-C₈)alkyl;

each R^{g} is, independently, H, (C₁-C₈)alkyl, or trifluoro-substituted (C₁-C₈)alkyl;

the molar ratio of y:z is 4:0.25-3; and

q has a value of 2 or less.

It is believed by the inventors herein that the key to this invention is the use of the molecule: {D(OR^{g})₄}_{y} as the second component of the reaction. In various aspects, the above reaction is carried out using the precursor {D(OR^{g})₄}_{y} and D is Si or Ti. In a further aspect, the above reaction is carried out using the precursor {D(OR^{g})₄}_{y} and D is Si. In a yet further aspect, the orthosilicates and orthotitanates used in the foregoing hydrolysis reaction may be Si(OCH₂CH₃)₄ or Ti(OCH(CH₃)₂)₄.

In a further aspect, the precursor silane:

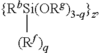

is a compound having the structure:

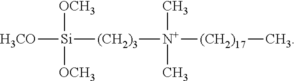

In a further aspect, the hydrolysis reaction is carried out with the y:z molar ratio of about 4:1-3. In a still further aspect, the hydrolysis reaction is carried out with the y:z molar ratio of about 4:1-2. In a yet further aspect, the hydrolysis reaction is carried out with the y:z molar ratio of about 4:1. In an even further aspect, the hydrolysis reaction is carried out with the y:z molar ratio of about 4:2. In a still further aspect, the hydrolysis reaction is carried out with the y:z molar ratio of about 4:0.5.

The (OR^{g}) group is selected from the group consisting of —OCH₃, —OCH₂CH₃, —OCH(CH₃)₂, —O(CH₂)₂CH₃, —OCH₂CH(CH₃)₂, —O(2-ethylhexyl), acetoxy, and, oximo. In a further aspect, the (OR^{g}) group is selected from the group consisting of —OCH₃, —OCH₂CH₃, and —OCH(CH₃)₂. In a further aspect, the (OR^{g}) group is selected from the group consisting of —OCH₃ and —OCH₂CH₃.

This hydrolysis is carried out using a stoichiometric or substantially stoichiometric amounts of water and a catalyst for hydrolysis and condensation. Stoichiometric amounts of water, or, an amount of water greater than stoichiometric, result in low molecular weight materials, which is one of the objectives of the method in this invention. Caution should be noted for the use of substantially lesser amounts of water as that will result in a residual amount of alkoxy in the material which is undesirable for purposes of this invention.

Stoichiometry is based on the number of hydrolysable groups on the combined components. The reaction is carried out in the presence of base or acid, with acid being the preferred catalyst. The acid catalysts are preferred to be HCl, phosphoric, and acetic acids, with HCl and phosphoric acids being most preferred.

Bases that are useable herein are amines, NaOH, KOH and the like and preferred for this invention is NaOH. The hydrolysis reaction is carried out by combining the components in a predetermined ratio and then adding acidic or basic water to the components at a controlled rate to form silanols from the alkoxy moieties. For some end use applications of the inventive materials, a slightly higher molecular weight (higher number of silanol reactive groups) is preferred and in this case, the silicate component is treated for a short period of time by acidic or basic water to cause the silicate component to hydrolyze and condense before the other components are added.

In various aspects, the disclosed pharmaceutical compositions comprising the compound of formula I, are useful in various methods of treating a disease or disorder in a subject that can be treated by administration of the pharmaceutical compositions. In one aspect, the disease or disorder treated is an infection. Accordingly, in various aspects, the invention is directed to a method of treating an infection in a subject, comprising the step(s) of administering to the subject an effective amount of the pharmaceutical composition comprising the compound of formula I.

In a further aspect of the method of treating an infection in a subject, comprising administering to the mammal an effective amount of the pharmaceutical composition comprising the compound of formula I, the administering step(s) is topical administration.

In a further aspect of the method of treating an infection in a subject, comprising administering to the mammal an effective amount of the pharmaceutical composition comprising the compound of formula I, the administering step(s) is inhalation or oral administration.

In a further aspect of the method of treating an infection in a subject, comprising administering to the mammal an effective amount of the pharmaceutical composition comprising the compound of formula I, the administering step(s) is intravenous or intra-arterial administration.

In a further aspect of the method of treating an infection in a subject, comprising administering to the mammal an effective amount of the pharmaceutical composition comprising the compound of formula I, the effective amount is a prophylactically effective amount.

In a further aspect of the method of treating an infection in a subject, comprising administering to the mammal an effective amount of the pharmaceutical composition comprising the compound of formula I, the effective amount is a therapeuticcally effective amount.

In a further aspect of the method of treating an infection in a subject, the subject is a bird. In a still further aspect, the bird is a domesticated bird. In a yet further aspect, the domesticated bird is a chicken, turkey, duck, or goose.

In a further aspect of the method of treating an infection in a subject, the subject is a mammal. In a still further aspect, the mammal is a human, a pig, a cow, a goat, a horse, a cat, or a dog. In a yet further aspect, the mammal is a human.

In a further aspect of the method of treating an infection in a subject, the subject has been diagnosed with a need for treatment of an infection prior to the administering step. In a still further aspect of the method of treating an infection in a subject, the subject has been diagnosed with conjunctivitis, keratitis, hepatitis, encephalitis, chickenpox, herpes, influenza, mumps, measles, viral meningitis, viral pneumonia, Ebola hemorrhagic fever, rubella, shingles, infectious mononucleosis, smallpox, gastroenteritis, AIDS, aspergillosis, blastomycosis, coccidioidomycosis, cryptococcal disease, candidiasis, histoplasmosis, sporotrichosis, or a combination thereof. In a still further aspect of the method of treating an infection in a subject, the subject has been diagnosed with conjunctivitis, keratitis, hepatitis, or encephalitis, or a combination thereof.

In a further aspect of the method of treating an infection in a subject, the subject has been diagnosed with Lyme's disease, granuloma inguinale, bacterial vaginosis, gonorrhea, syphilis, congenital syphilis, Mycobacterium avium complex, melioidosis, anthrax, leptospirosis, whooping cough, leprosy, tetanus, plague, bubonic plague, pneumonic plague, scarlet fever, streptococcal infection, invasive group a streptococcal disease, streptococcal toxic shock syndrome, meningococcal disease, bacteremia, strep throat, cholera, dysentery, amebic dysentery, shigellosis, diphtheria, cutaneous diphtheria, respiratory diphtheria, legionnaires' disease, tuberculosis, latent tuberculosis, Hemophilus influenzae B, typhoid fever, Rocky Mountain spotted fever, Vibrio parahaemolyticus, Vibrio vulnificus, Vibrio, yersiniosis, whipple's disease, bacterial digestive infection, acute appendicitis—meningitis, bacterial meningitis, encephalitis, impetigo, cellulitis, carbuncle, boil, acne, sepsis, septicemia, pneumonia, ptomaine food poisoning, Salmonella food poisoning, Salmonella enteritidis, staphylococcal infection, Staphylococcus aureus food poisoning, botulism food poisoning, infant botulism food poisoning, E. coli food poisoning, rheumatic fever, brucellosis, ehrlichiosis, psittacosis, acanthamoeba, granulomatous amebic encephalitis, relapsing fever, naegleria, diarrheagenic Escherichia coli, listeriosis, scombrotoxic fish poisoning, trachoma, Chlamydia pneumoniae, Mycoplasma pneumoniae, mycobacterial infections, q fever, stari, yaws, actinomycosis, lymphogranuloma venereum, bacterial toxins—fetal exposure, Helicobacter pylori infection, Legionella adelaidensis infection, Legionella anisa infection, Legionella beliardensis infection, Legionella birminghamensis infection, Legionella bozemanii infection, Legionella bruneiensis infection, Legionella brunensis infection, Legionella busanensis infection, Legionella cherrii infection, Legionella cincinnatiensis infection, Legionella donaldsonii infection, Legionella donaldsonil infection, Legionella drancourtii infection, Legionella drozanskii infection, Legionella dumofii infection, Legionella erythra infection, Legionella fairfieldensis infection, Legionella fallonii infection, Legionella feelei infection, Legionella feeleii infection, Legionella gesstiana infection, Legionella gormanii infection, Legionella gratiana infection, Legionella gresilensis infection, Legionella hackeliae infection, Legionella impletisoli infection, Legionella isrealensis infection, Legionella jamestowniensis infection, Legionella jordanis infection, Legionella lansingensis infection, Legionella londinensis infection, Legionella lytica infection, Legionella maceachemii infection, Legionella maceachernii infection, Legionella micdadei infection, Legionella monrovica infection, Legionella moravica infection, Legionella nautarum infection, Legionella oakridgensis infection, Legionella parisiensis infection, Legionella quateirensis infection, Legionella quinlivanii infection, Legionella rowbothamii infection, Legionella rubrilucens infection, Legionella sainthelensi infection, Legionella santicrucis infection, Legionella shakespearei infection, Legionella spiritensis infection, Legionella steigerwaltii infection, Legionella tauriensis infection, Legionella tusconensis infection, Legionella wadsorthii infection, Legionella wadsworthii infection, Legionella waltersii infection, Legionella worsliensis infection, Legionella yabuuchiae infection, Salmonella anatum infection, Salmonella choleraesuis infection, Salmonella enteritidis infection, Salmonella heidelberg infection, Salmonella hirschfeldii infection, Salmonella newport infection, Salmonella paratyphi a infection, Salmonella schottmuelleri infection, Salmonella typhi infection, Salmonella typhimurium infection, Shigella boydii infection, Shigella dysenteriae infection, Shigella flexneri infection, Shigella sonnei infection, Vibrio infection—Vibrio cincinnatiensis, Vibrio infection—Vibrio damsela, Vibrio infection—Vibrio fluvialis, Vibrio infection—Vibrio furnissii, Vibrio infection—Vibrio holisae, Vibrio infection—Vibrio metschnikovii, Vibrio infection—Vibrio mimicus, enteroaggregative E. coli infection, enterohemorrhagic E. coli infection, enteroinvasive E. coli infection, enteropathogenic E. coli infection, enterotoxigenic E. coli infection, cheese washer's lung—Penicillium spp., farmer's lung—Thermoactinomyces vulgaris, syphilitic aseptic meningitis, actinomycotic appendicitis, bacterial appendicitis, Campylobacter jejuni subspecies doylei infection, Campylobacter laridis infection, Campylobacter sputorum infection, Campylobacter food poisoning, clostridium perfringens food poisoning, bacterial conjunctivitis, Pneumococcal meningitis, bacterial septicemia, acute bacterial prostatitis, chronic bacterial prostatitis, small bowel bacterial overgrowth syndrome, Bacillus cereus type I food poisoning, Bacillus cereus type II food poisoning, bacterial pericarditis, humidifier lung—Bacillus spp., prostatic tuberculosis, bacterial prostatitis, renal tuberculosis, anthrax meningitis, meningococcal a, meningococcal b, meningococcal c, post-streptococcal glomerulonephritis, Chlamydia, mastitis, bartholin's abscess, Chlamydial infection, acute tracheitis, cryptosporiosis, pneumonia, bacterial pneumonia, staphylococcal pneumonia, Pseudomonas aeruginosa, neonatal bacterial meningitis, cryptococcosis, drug-resistant Streptococcus pneumoniae disease, drug-resistant Streptococcus pneumoniae, glanders, nocardiosis, sporotrichosis, Mycobacterium bovis, Mycobacterium kansasii, Mycobacterium xenopi, Mycobacterium scrofulaceum, Mycobacterium abscessus, Mycobacterium haemophilum, Mycobacterium ulcerans, bacterial endocarditis, erythrasma, epiglotitis, Pneumococcal pneumonia, Pneumococcus, acute rheumatic fever, pemphigus neonatorum, erysipeloid, erysipelas, barber's rash, tuberculous pericarditis, pyogenic pericarditis, tracheitis, serratia meningitis, vaginosis (bacterial vaginosis), listeriosis meningoencephalitis, neurosyphilis, Mycobacterium tuberculosis, cryptococcal meningitis, cutaneous anthrax, pulmonary anthrax, gastrointestinal anthrax, tularemia, bacteriuria, Streptococcal group A invasive disease, serratia urinary tract infection, Edwardsiella tarda infection, bortonneuse fever, malignant buotonneuse fever, Eikenella corrodens infection, necrobacillosis, Vibrio mimicus food poisoning, typhus, paratyphoid fever, epidemic typhus, murine typhus, brill-zinsser disease, recrudescent typhus, kenya tick typhus, scrub typhus, queensland tick typhus, chancroid, ureaplasma urealyticum, primary syphilis, secondary syphilis, tertiary syphilis, Burkholderia pseudomallei, Pseudomonas pseudomallei, Weil's Syndrome, nanukayami, cephalic tetanus, neonatal tetanus, group a streptococcal infections, group b Streptococcal infections, necrotizing fasciitis, meningococcemia, Shigella flexneri, Shigella boydii, Shigella sonnei, Pontiac fever, tuberculous meningitis, listeriosis sepsis, post-streptococcal neurologic disorders, staphylococcal food poisoning, mountain fever, mountain tick fever, Marseilles fever, Kenya fever, Indian tick fever, Conor's disease, Bruch's disease, escharonodulaire, Kenya tick-bite fever, India tick typhus, Israeli spotted fever, Boutonneuse fever, Helicobacter pylori bacteria, capnocytophaga, dermatophilosis, Francisella tularenis infection, Helicobacter fenneliae infection, Serratia ear infection, Bartonella infections, Fournier gangrene, tuberculous uveitis, pinta, spotted fevers, Mediterranean spotted fever, enterotoxigenic Escherichia coli, enterohemorrhagic Escherichia coli infection, Campylobacter jejuni, rheumatic heart disease, toxic shock syndrome, Campylobacter fetus infection, Pseudomonas infections, arcobacter butzleri infection, Arcobacter cryaerophilus infection, Arcobacter infection, Vibrio vulnificus infection, Treponema infection, Moraxella catarrhalis infection, infection with Mycobacterium marinum, meningococcal infection, Pseudomonas stutzeri infections, Mycobacterium avium complex infection, Actinomycetales infection, disseminated infection with Mycobacterium avium complex, African tick typhus, bartonellosis due to Bartonella quintana infection, serratia respiratory tract infection, Bacillaceae infections, Legionella longbeachae infection, Helicobacter cinaedi infection, constrictive tuberculous pericarditis, colibacillosis, Campylobacter hylointestinalis infection, Campylobacter jejuni infection, scarletina (scarlet fever), sennetsu fever, spirochetes disease, bartonellosis, rickettsia, the clap, honeymoon bladder, Clostridium sordellii, Serratia, Serratia sepsis, Serratia cerebral abscess, rhodococcus equi, bacterial toxic-shock syndrome, streptococcal group B invasive disease, Rickettsia siberica, sporotrichosis—pulmonary, rickettsial disease, syphilis, latent, rickettsia typhi, ricketttsialpox, listeriosis of pregnancy, urosepsis, gonococcal urethritis, bartonella, vancomycin resistant enterococcal bacteremia, congenital tuberculosis, neurosyphilis, Mycobacterium fortuitum, mendelian susceptibility to atypical mycobacteria, yersinia pseudotuberculosis, listeriosis—granulomatous infantiseptica, borreliosis, neurosyphilis—asymptomatic, human monocytic ehrlichiosis, staphylococcal toxic shock syndrome, neurosyphilis tabes dorsalis, lysteria monocytoigeneses meningitis, pyomyositis, pasteurella multocida, tuberculosis, pulmonary, erythema chronicum migrans, neurosyphilis meningovascular, hidradenitis suppurativa, weil syndrome, flavimonas oryzihabitans, bar's syndrome, austrian syndrome, brill disease, stenotrophomonas maltophilia, bejel, human granulocytic ehrlichiosis, waterhouse-friederichsen syndrome, durand-nicolas-favre syndrome, ausrian triad, malaria, Entamoeba spp. infection, Plasmodiumi spp. infection, Giardia spp. infection, Trypanosoma spp. infection, Balantidium spp. infection, Trichomonas spp. infection, Cryptosporidiium spp. infection, Isopora spp. infection, Entamoeba histolytica infection, Balantidium coli infection, Giardia lamblia infection, Trichomonas vaginalis infection, Cryptosporidium parvum infection, Isospora belli infection, Plasmodium falciparum infection, Plasmodium vivax infection, Plasmodium ovale infection, Plasmodium malariae infection, Plasmodium knowlesi infection, Plasmodium brasilianum infection, Plasmodium cynomolgi infection, Plasmodium cynomolgi bastianeffii infection, Plasmodium inui infection, Plasmodium rhodiani infection, Plasmodium schweitzi infection, Plasmodium semiovale infection, Plasmodium simium infection, or a combination thereof.

In a further aspect of the method of treating an infection in a subject, the method further comprises the step of identifying a subject in need of treatment of an infection.

In a further aspect of the method of treating an infection in a subject, the infection is caused by a microorganism or a virus.

In a further aspect of the method of treating an infection in a subject, the infection is caused by a microorganism. In a still further aspect of the method of treating an infection in a subject, the infection is caused by microorganism; and the microorganism is a bacterium. In a yet further aspect of the method of treating a bacterial infection in a subject, the infection is caused by Escherichia coli, Staphylococcus aureus, Chlamydia trachomatis, Porphyromonas gingivalis, or a combination thereof. In an even further aspect of the method of treating a bacterial infection in a subject, the infection is caused by Acetobacter aurantius, Acinetobacter baumannii, Actinomyces israelii, Agrobacterium radiobacter, Agrobacterium tumefaciens, Anaplasma phagocytophilum, Azorhizobium caulinodans, Azotobacter vinelandii, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacteroides fragilis, Bacteroides gingivalis, Bartonella henselae, Bartonella quintana, Bordetella bronchiseptica, Bordetella pertussis, Borrelia burgdorferi, Brucella abortus, Brucella melitensis, Brucella suis, Burkholderia mallei, Burkholderia pseudomallei, Burkholderia cepacia, Calymmatobacterium granulomatis, Campylobacter coli, Campylobacter fetus, Campylobacter jejuni, Campylobacter pylori, Chlamydia trachomatis, Chlamydophila pneumoniae, Chlamydophila psittaci, Clostridium botulinum, Clostridium difficile, Clostridium perfringens, Clostridium tetani, Corynebacterium diphtheriae, Corynebacterium fusiforme, Coxiella bumetiii, Ehrlichia chaffeensis, Enterobacter cloacae, Enterococcus avium, Enterococcus durans, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Lactobacillus acidophilus, Lactobacillus bulgaricus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Neisseria gonorrhoeae, Neisseria meningitidis, Pasteurella multocida, Pasteurella tularensis, Peptostreptococcus, Porphyromonas gingivalis, Prevotella melaninogenica, Pseudomonas aeruginosa, Rhizobium radiobacter, Rickettsia prowazekii, Rickettsia psittaci, Rickettsia quintana, Rickettsia rickettsia, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Staphylococcus aureus, Staphylococcus epidermidis, Stenotrophomonas maltophilia, Streptococcus agalactiae, Streptococcus avium, Streptococcus bovis, Streptococcus cricetus, Streptococcus faecium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema pallidum, Treponema denticola, Vibrio cholerae, Vibrio comma, Vibrio parahaemolyticus, Vibrio vulnificus, Wolbachia, Yersinia enterocolitica, Yersinia pestis, Yersinia pseudotuberculosis, or a combination thereof.

In a further aspect of the method of treating an infection in a subject, the infection is caused by microorganism; and the microorganism is a fungus. In a yet further aspect of the method of treating a fungal infection in a subject, the infection is caused by Aspergillus fumigatusi, Aspergillus flavus, Candida albicans, Candida ascalaphidarum, Candida amphixiae, Candida antarctica, Candida argentea, Candida atlantica, Candida atmosphaerica, Candida blattae, Candida bromeliacearum, Candida carpophila, Candida carvajalis, Candida cerambycidarum, Candida chauliodes, Candida corydali, Candida dosseyi, Candida dubliniensis, Candida ergatensis, Candida fructus, Candida glabrata, Candida fermentati, Candida guilliermondii, Candida haemulonii, Candida insectamens, Candida insectorum, Candida intermedia, Candida jeffresii, Candida kefyr, Candida keroseneae, Candida krusei, Candida lusitaniae, Candida lyxosophila, Candida maltosa, Candida marina, Candida membranifaciens, Candida milleri, Candida mogii, Candida oleophila, Candida oregonensis, Candida parapsilosis, Candida quercitrusa, Candida rugosa, Candida sake, Candida shehatea, Candida temnochilae, Candida tenuis, Candida theae, Candida tolerans, Candida tropicalis, Candida tsuchiyae, Candida sinolaborantium, Candida sojae, Candida subhashii, Candida viswanathii, Candida utilis, Candida ubatubensis, Candida zemplinina, Cryptococcus laurentii, Cryptococcus neoformans, Cryptococcus albidus, Cryptococcus gatti, Histoplasma capsulatum, Pneumocystis jirovecii, Pneumocystis carinii, Stachybotrys chartarum, or a combination thereof.

In a further aspect of the method of treating an infection in a subject, the infection is caused by a microorganism; and the microorganism is a protozoan. In a still further aspect of the method of treating a protozoan infection in a subject, the infection is caused by Entamoeba spp., Plasmodiumi spp., Giardia spp., Trypanosoma spp., Balantidium spp., Trichomonas spp., Cryptosporidiium spp., Isopora spp., or a combination thereof. In a yet further aspect of the method of treating a protozoan infection in a subject, the infection is caused by Entamoeba histolytica, Balantidium coli, Giardia lamblia, Trichomonas vaginalis, Cryptosporidium parvum, Isospora belli, Plasmodium falciparum, Plasmodium vivax, Plasmodium ovale, Plasmodium malariae, Plasmodium knowlesi, Plasmodium brasilianum, Plasmodium cynomolgi, Plasmodium cynomolgi bastianeffii, Plasmodium inui, Plasmodium rhodiani, Plasmodium schweitzi, Plasmodium semiovale, Plasmodium simium, or a combination thereof. In an even further aspect of the method of treating a protozoan infection in a subject, the infection is caused by Entamoeba histolytica, Balantidium coli, Giardia lamblia, Trichomonas vaginalis, Cryptosporidium parvum, Isospora belli, or combination thereof. In a still further aspect of the method of treating a protozoan infection in a subject, the infection is caused by Plasmodium falciparum, Plasmodium vivax, Plasmodium ovale, Plasmodium malariae, Plasmodium knowlesi, Plasmodium brasilianum, Plasmodium cynomolgi, Plasmodium cynomolgi bastianellii, Plasmodium inui, Plasmodium rhodiani, Plasmodium schweitzi, Plasmodium semiovale, Plasmodium simium, or a combination thereof.

In a further aspect of the method of treating an infection in a subject, the infection is caused by a virus. In a still further aspect of the method of treating an infection in a subject, the infection is caused by a virus; and the virus is a human DNA virus. In a still further aspect of the method of treating an infection in a subject, the infection is caused by a virus; and the virus is a human RNA virus. In a yet further aspect of the method of treating an infection in a subject, the infection is caused by a virus; and the virus is an ebola virus, a paramyxovirus, a parainfluenza virus, a morbillivirus, an immunodeficiency virus, a respovirus, a rubalavirus, varicella-zoster virus, a variola virus, a herpesvirus, an influenza virus, a pneumovirus, a metapneumovirus, a rubivirus, an astrovirus, an enteric adenovirus, a norovirus, a rotavirus, a hepatitis virus, an arbovirus, an Epstein-Barr virus, an enterovirus, a coxsackievirus, an echovirus, or a combination thereof. In an even further aspect of the method of treating an infection in a subject, the infection is caused by a virus; and the virus is feline calicivirus, herpes virus, or a combination thereof.

In a further aspect of the method of treating an infection in a subject, the infection is caused by ebola virus.

In a further aspect of the method of treating an infection in a subject, the infection is caused by a virus; and the virus is a herpes virus selected from the group consisting of HSV-1, HSV-2, HHV-6, HHV-7, HHV-8, HCMV, EBV, VZV, or a combination thereof. In a still further aspect of the method of treating an infection in a subject, the infection is caused by a virus; and the virus is a herpes virus selected from the group consisting of HSV-1, HHV-6A, HHV-6B, HHV-7, HCMV, EBV, or a combination thereof. In a yet further aspect of the method of treating an infection in a subject, the infection is caused by a herpes virus, and the herpes virus is HSV-1. In an even further aspect of the method of treating an infection in a subject, the infection is caused by a herpes virus, and the herpes virus is HSV-2.

In a further aspect of the method of treating an infection in a subject, the infection is caused by a virus; and the virus is an influenza virus. In a still further aspect of the method of treating an infection in a subject, the infection is caused by a virus; and the virus is an influenza virus is a type A influenza virus, type B influenza virus, and type C influenza virus. In a yet further aspect of the method of treating an infection in a subject, the infection is caused by a virus; and the virus is an influenza virus is a type A influenza virus. In an even further aspect of the method of treating an infection in a subject, the infection is caused by an influenza virus; and the influenza virus is an influenza virus is H1N1, H1N2, H2N2, H3N2, H3N8, H5N1, H5N2, H5N3, H5N8, H5N9, H7N1, H7N2, H7N3, H7N4, H7N7, H9N2, or H10N7.

In a further aspect of the method of treating an infection in a subject, the infection is caused by a virus; and the virus is an immunodeficiency virus. In a still further aspect of the method of treating an infection in a subject, the infection is caused by an immunodeficiency virus; and the immunodeficiency virus is HIV. In a yet further aspect of the method of treating an infection in a subject, the infection is caused by HIV; and the HIV is HIV-1 serotype virus. In an even further aspect, the HIV-1 serotype virus is selected from the group consisting Group M, Group N, Group O, Group P virus strain, or combinations thereof.

In a further aspect of the method of treating an infection in a subject, the infection is associated with biological dark matter. In a still further aspect, of the method of treating an infection in a subject, the method further comprises the step of determining in a subject the presence of genetic material that is biological dark matter and is not associated with a bacterium, an archaea organism, and a eukaryote. In a yet further aspect, of the method of treating an infection in a subject, the method further comprises the steps of (a) determining in a subject the presence of genetic material that is biological dark matter and is not associated with a bacterium, an archaea organism, and a eukaryote; and (b) determining whether the presence of genetic material that is biological dark matter decreases upon administration of a pharmaceutical composition comprising a compound of formula I.

In one aspect, the disease or disorder treated by administration of the disclosed pharmaceutical compositions comprising the compound of formula I is pain. Accordingly, in various aspects, the invention is directed to a method of reducing pain associated with a cutaneous or mucosal membrane lesion caused by a herpes viral infection in a mammal, comprising administering to the mammal an effective amount of the pharmaceutical composition comprising the compound of formula I.

In a further aspect of the method to reduce pain, the cutaneous or mucosal membrane lesion is located in the mouth, lips, nose, eye, gums, conjunctiva, cornea, ear, lung, genitalia, urethra, rectum, colon, sensory ganglia, or a combination thereof.

In one aspect, the disease or disorder treated by administration of the disclosed pharmaceutical compositions comprising the compound of formula I is a wound. Accordingly, in various aspects, the invention is directed to a method of healing a cutaneous or mucosal membrane lesion caused by a herpes viral infection in a mammal, comprising administering to the mammal an effective amount of the pharmaceutical composition comprising the compound of formula I.

In a further aspect of the method of healing, the cutaneous or mucosal membrane lesion is located in the mouth, lips, nose, eye, gums, conjunctiva, cornea, ear, lung, genitalia, urethra, rectum, colon, sensory ganglia, or a combination thereof.

In various aspects, the invention is directed to a method of treating a disorder of uncontrolled cellular proliferation in a subject, comprising the step(s) of administering to the subject an effective amount of the pharmaceutical composition comprising the compound of formula I.

In a further aspect of the method of treating a disorder of uncontrolled cellular proliferation in a subject, comprising administering to the mammal an effective amount of the pharmaceutical composition comprising the compound of formula I, the administering step(s) is oral administration.

In a further aspect of the method of treating a disorder of uncontrolled cellular proliferation in a subject, comprising administering to the mammal an effective amount of the pharmaceutical composition comprising the compound of formula I, the administering step(s) is intravenous or intra-arterial administration.

In a further aspect of the method of treating a disorder of uncontrolled cellular proliferation in a subject, comprising administering to the mammal an effective amount of the pharmaceutical composition comprising the compound of formula I, the effective amount is a prophylactically effective amount.

In a further aspect of the method of treating a disorder of uncontrolled cellular proliferation in a subject, comprising administering to the mammal an effective amount of the pharmaceutical composition comprising the compound of formula I, the effective amount is a therapeutically effective amount.

In a further aspect of the method of treating a disorder of uncontrolled cellular proliferation in a subject, the subject is a mammal. In a still further aspect, the mammal is a human.

In a further aspect of the method of treating a disorder of uncontrolled cellular proliferation in a subject, the method further comprises the step of identifying a subject in need of treatment of a disorder of uncontrolled cellular proliferation.

In a further aspect of the method of treating a disorder of uncontrolled cellular proliferation in a subject, the subject has been diagnosed with a need for treatment of a disorder of uncontrolled cellular proliferation prior to the administering step.

In a further aspect of the method of treating a disorder of uncontrolled cellular proliferation in a subject, the subject has been diagnosed with a cancer. In a still further aspect of the method of treating a disorder of uncontrolled cellular proliferation in a subject, the subject has been diagnosed with a cancer, and the cancer is a hematological cancer. In a still further aspect, the hematological cancer is a leukemia, lymphoma, chronic myeloproliferative disorder, myelodysplastic syndrome, myeloproliferative neoplasm, plasma cell neoplasm (myeloma), solid tumor, sarcoma, or carcinoma.

In a further aspect of the method of treating a disorder of uncontrolled cellular proliferation in a subject, the subject has been diagnosed with a cancer, the cancer is a hematological cancer, and the hematological cancer is leukemia. In a still further aspect, the leukemia is acute leukemia, acute lymphocytic leukemia, acute myelocytic leukemia, myeloblastic leukemia, promyelocytic leukemia, myelomonocytic leukemia, monocytic leukemia, erythroleukemia, chronic leukemia, chronic myelocytic (granulocytic) leukemia, or chronic lymphocytic leukemia.

In a further aspect of the method of treating a disorder of uncontrolled cellular proliferation in a subject, the subject has been diagnosed with a cancer, the cancer is a hematological cancer, and the hematological cancer is lymphoma. In a still further aspect, the lymphoma is AIDS-Related lymphoma, cutaneous T-Cell lymphoma, Hodgkin lymphoma, non-Hodgkin lymphoma, primary central nervous system lymphoma, mycosis fungoides and the Sézary Syndrome, heavy chain disease, or Waldenström macroglobulinemia. In a yet further aspect, the lymphoma is Hodgkin's lymphoma or non-Hodgkin's lymphoma.

In a further aspect of the method of treating a disorder of uncontrolled cellular proliferation in a subject, the subject has been diagnosed with a cancer, and the cancer is a sarcoma. In a still further aspect, the sarcoma is a fibrosarcoma, myxosarcoma, liposarcoma, chondrosarcoma, osteogenic sarcoma, chordoma, angiosarcoma, endotheliosarcoma, lymphangiosarcoma, leiomyosarcoma, rhabdomyosarcoma, or lymphangioendotheliosarcoma.

In a further aspect of the method of treating a disorder of uncontrolled cellular proliferation in a subject, the subject has been diagnosed with a cancer, and the cancer is a carcinoma. In a still further aspect, the carcinoma is a colon carcinoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, bile duct carcinoma, choriocarcinoma, seminoma, embryonal carcinoma, lung carcinoma, small cell lung carcinoma, bladder carcinoma, or epithelial carcinoma.

In a further aspect of the method of treating a disorder of uncontrolled cellular proliferation in a subject, the subject has been diagnosed with a cancer, and the cancer is a synovioma, mesothelioma, Ewing's tumor, pancreatic cancer, breast cancer, ovarian cancer, prostate cancer, hepatoma, Wilms' tumor, cervical cancer, testicular cancer, glioma, astrocytoma, medulloblastoma, craniopharyngioma, ependymoma, pinealoma, or retinoblastoma.

In a further aspect of the method of treating a disorder of uncontrolled cellular proliferation in a subject, the subject has been diagnosed with a cancer, and the cancer is a cancer of brain, genitourinary tract, gastrointestinal tract, colon, rectum, breast, kidney, lymphatic system, stomach, lung, pancreas, skin, or combination thereof.

In a further aspect of the method of treating a disorder of uncontrolled cellular proliferation in a subject, the subject has been diagnosed with a cancer, and the cancer is prostate cancer, glioblastoma multiforme, endometrial cancer, breast cancer, colon cancer, or a combination thereof.

In various aspects, the invention is directed to a method for inducing apoptosis in a subject, comprising the step(s) of administering to the subject an effective amount of the pharmaceutical composition comprising the compound of formula I. In a further aspect, the invention is directed to a method for inducing apoptosis in a subject, comprising the step(s) of administering to the subject an effective amount of the pharmaceutical composition comprising the compound of formula I, thereby treating a disorder of uncontrolled cellular proliferation.

In a further aspect of the method for inducing apoptosis in a subject, comprising administering to the mammal an effective amount of the pharmaceutical composition comprising the compound of formula I, the effective amount is a prophylactically effective amount.

In a further aspect of the method for inducing apoptosis in a subject, comprising administering to the mammal an effective amount of the pharmaceutical composition comprising the compound of formula I, the effective amount is a therapeutically effective amount.

In a further aspect of the method for inducing apoptosis in a subject, the subject is a mammal. In a still further aspect, the mammal is a human.

In a further aspect of the method for inducing apoptosis in a subject, the method further comprises the step of identifying a subject in need of inducing apoptosis.

In a further aspect of the method for inducing apoptosis in a subject, the subject has been diagnosed with a need for inducing apoptosis prior to the administering step. In a still further aspect of the method for inducing apoptosis in a subject, the subject has been diagnosed with a need for inducing apoptosis prior to the administering step, and the need for inducing apoptosis is associated with treatment of a disorder of uncontrolled cellular proliferation. In a still further aspect of the method for inducing apoptosis in a subject, the subject has been diagnosed with a need for inducing apoptosis prior to the administering step, the need for inducing apoptosis is associated with treatment of a disorder of uncontrolled cellular proliferation, and the disorder of uncontrolled cellular proliferation is a cancer.

In various aspects, the invention is directed to a method for inducing apoptosis in at least one cell, the method comprising the step of contacting the at least one cell with an effective amount of at least one compound of formula I or a pharmaceutical composition comprising formula I.

In a further aspect of the method for inducing apoptosis in at least one cell, the at least one cell is in subject and contacting the cell is via administration of the least one compound of formula I or the pharmaceutical composition comprising formula Ito a subject.

In a further aspect of the method for inducing apoptosis in at least one cell, the at least one cell is a mammalian cell and the mammalian cell is ex vivo.

In various aspects, the disclosed pharmaceutical compositions comprising the compound of formula I, are useful in various kits useful in the medical arts. Accordingly, in various aspects, the invention is directed to a kit comprising the disclosed pharmaceutical compositions comprising the compound of formula I, and at least one of:
a) at least one therapeutic agent known to treat a viral infection;
b) at least one therapeutic agent known to treat a bacterial infection;
c) instructions for treating a viral infection;
d) instructions for treating a bacterial infection;
e) instructions for administering the pharmaceutical composition in connection with treating a viral infection; or
f) instructions for administering the pharmaceutical composition in connection with treating a bacterial infection.

In a further aspect of the kit comprising the disclosed pharmaceutical compositions comprising the compound of formula I, the pharmaceutical composition and the at least one therapeutic agent known to treat a viral infection are co-packaged.

In a further aspect of the kit comprising the disclosed pharmaceutical compositions comprising the compound of formula I, the pharmaceutical composition and the at least one therapeutic agent known to treat a viral infection are co-formulated.

In a further aspect of the kit comprising the disclosed pharmaceutical compositions comprising the compound of formula I, the pharmaceutical composition and the at least one therapeutic agent known to treat a bacterial infection are co-packaged.

In a further aspect of the kit comprising the disclosed pharmaceutical compositions comprising the compound of formula I, the pharmaceutical composition and the at least one therapeutic agent known to treat a bacterial infection are co-formulated.

It is also contemplated that any one or more species can be optionally omitted from the genus of formula I described herein above. It is understood that a disclosed compound of formula I can be prepared as described herein above and/or methods known to one skilled in the art. It is also understood that the disclosed pharmaceutical compostions comprising a compound of formula I can be employed in the disclosed methods, medicaments, uses, and kits disclosed herein.

In various aspects, the pharmaceutical compositions of the present invention may be provided comprising an effective amount of at least one disclosed compound.

In a further aspect, the effective amount is a therapeutically effective amount. In a still further aspect, the effective amount is a prophylactically effective amount.

In a further aspect, the pharmaceutical composition comprises a disclosed compound. In a yet further aspect, the pharmaceutical composition comprises a product of a disclosed method of making.

In certain aspects, the disclosed pharmaceutical compositions comprise the disclosed compounds as an active ingredient, a pharmaceutically acceptable excipient, and, optionally, other therapeutic carriers, ingredients, and/or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In practice, the compounds of the invention may be combined as the active ingredient in intimate admixture with a pharmaceutical excipient according to conventional pharmaceutical compounding techniques. The excipient can take a wide variety of forms depending on the form of preparation desired for administration, e.g., oral or parenteral (including intravenous). Thus, the pharmaceutical compositions of the present invention may be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the pharmaceutical compositions can also be administered by controlled release means and/or delivery devices. The pharmaceutical compositions can be prepared by any of the methods of pharmacy. In general, such methods include a step of bringing into association the active ingredient, i.e., a disclosed compound, with the excipient that constitutes one or more necessary ingredients. In general, the compositions are prepared by uniformly and intimately admixing the active ingredient with liquid excipients or finely divided solid excipients or both. The product can then be conveniently shaped into the desired presentation.

Thus, the pharmaceutical compositions of this invention may further comprise at least one other therapeutic agent.

The pharmaceutical excipient that employed may be, for example, any solid, liquid, semi-solid or, in the case of an aerosol composition, gaseous excipient that is generally available to one of skill in the art. Examples of solid excipients include, but are not limited to, starch, cellulose, hydroxypropyl cellulose, glucose, lactose, gelatin, malt, rice, flour, chalk, silica gel, sodium chloride, dried skim milk, terra alba, sucrose, talc, gelatin, agar, pectin, acacia, glycerol monostearate, magnesium stearate, sodium stearate, and stearic acid. Examples of liquid excipients include, but are not limited to, acetone, glycerol, dimethylsulfoxide (“DMSO”), ethanol, 1,3-propanediol, propylene glycol, sugar syrup, water, and various oils, including those of petroleum, animal, vegetable or synthetic origin, e.g., olive oil, peanut oil, soybean oil, mineral oil, and sesame oil. Further examples of liquid excipients are a solvent disclosed herein, that is, a solvent such as acetic acid, acetone, anisole, 1,2-butanediol, 1,3-butanediol, 1,4-butanediol, 1-butanol, 2-butanol, dimethyl sulfoxide, ethanol, ethyl acetate, ethyl ether, ethyl formate, formic acid, heptane, isobutyl acetate, isopropyl acetate, methyl acetate, 3-methyl-1-butanol, butyl acetate, methylethyl ketone, tert-butylmethyl ether, methylisobutyl ketone, 2-methyl-1-propanol, pentane, 1-pentanol, 1-propanol, 2-propanol, propyl acetate, or a combination thereof.

Examples of gaseous excipients include, but are not limited to, carbon dioxide and nitrogen.

In various aspects, the compositions provided herein may also include one or more of a-tocopherol, gum arabic, and/or hydroxypropyl cellulose.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while excipients such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical excipients are employed. Optionally, tablets can be coated by standard aqueous or nonaqueous techniques

A tablet comprising the pharmaceutical composition of this invention may be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The excipient can be a solvent or dispersion medium containing, for example, acetone, DMSO, ethanol, polyol (e.g., glycerol, propylene glycol and liquid polyethylene glycol), 1,3-propanediol, vegetable oils, water, and suitable mixtures thereof.

Pharmaceutical compositions of the present invention may be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention may be in a form suitable for rectal administration wherein the excipient is a solid. It is preferable that the mixture forms unit dose suppositories. Suitable excipients include cocoa butter and other materials commonly used in the art. The suppositories can be conveniently formed by first admixing the pharmaceutical composition with the softened or melted excipient(s) followed by chilling and shaping in molds.

In addition to the aforementioned excipient ingredients, the pharmaceutical formulations described above can include, as appropriate, one or more additional excipient ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In the treatment conditions which require a pharmaceutical composition for treatment of an infection, an appropriate dosage level of a disclosed compound will generally be about 0.01 to 500 mg per kg patient body weight per day and can be administered in single or multiple doses. Preferably, the dosage level will be about 0.1 to about 250 mg/kg per day; more preferably 0.5 to 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 milligrams of the active ingredient, particularly 1.0, 5.0, 10, 15, 20, 25, 50, 75, 100, 150, 200, 250, 300, 350, 400, 450, 500, 550, 600, 650, 700, 750, 800, 850, 900, 950, and 1000 milligrams of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

It is understood, however, that the specific dose level for any particular patient will depend upon a variety of factors. Such factors include the age, body weight, general health, sex, and diet of the patient. Other factors include the time and route of administration, rate of excretion, drug combination, and the type and severity of the particular disease undergoing therapy.

The disclosed pharmaceutical compositions can further comprise other therapeutically active compounds, which are usually applied in the treatment of the above mentioned pathological conditions.

It is understood that the disclosed compositions can be prepared from the disclosed compounds. It is also understood that the disclosed compositions can be employed in the disclosed methods of using.

The present invention is further defined in the following Examples, in which all parts and percentages are by weight, unless otherwise stated. It should be understood that these examples, while indicating preferred embodiments of the invention, are given by way of illustration only and are not to be construed as limiting in any manner. From the above discussion and these examples, one skilled in the art can ascertain the essential characteristics of this invention, and without departing from the spirit and scope thereof, can make various changes and modifications of the invention to adapt it to various usages and conditions.

### EXAMPLES

### Example 1

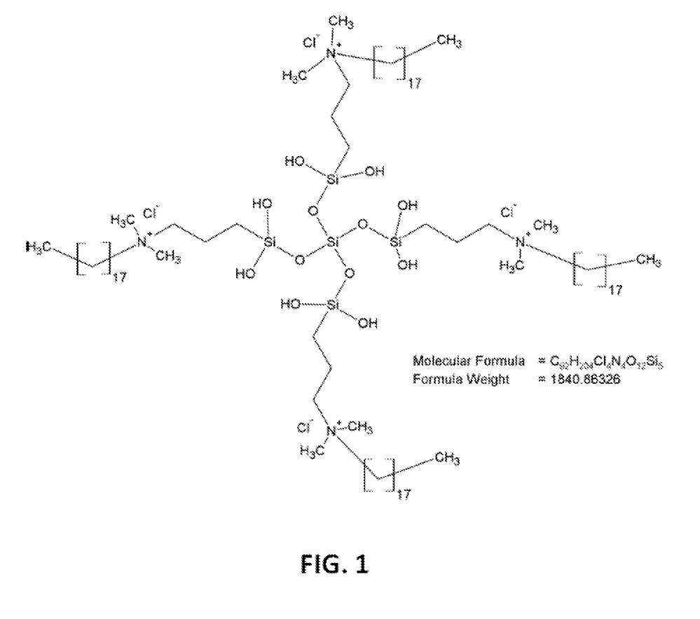

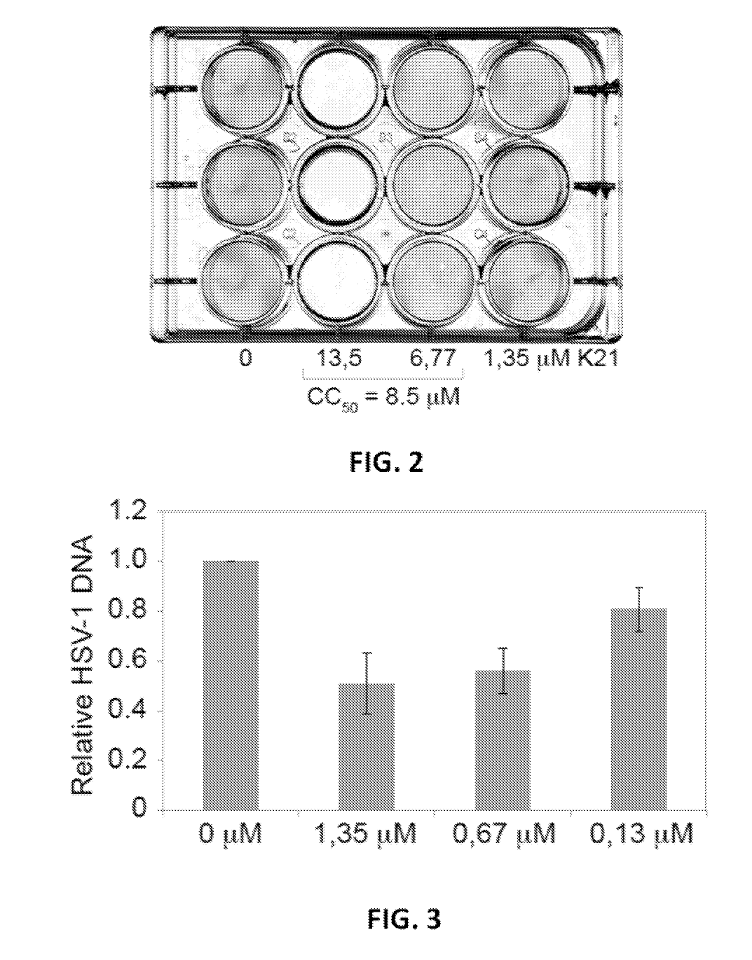

The 50% cytotoxic concentration (CC₅₀) of a representative disclosed compound, K-21, was determined in Vero cells, and the aata are shown in FIG. 2. The structure of K-21 is shown in FIG. 1. The growth of Vero cells was carried out under standard conditions. The data show that K-21 has a CC₅₀ of about 8.5 μM.

### Example 2

The inhibition of HSV-1 infection in Vero cells was determined by a exposing cells infected cells to compound K-21, and the data are shown in FIG. 3. The HSV-1 strain used in the experiment is HSV-1 strain F (ATCC VR-733). The data shown in FIG. 3 show that K-21 at a concentration of 1.35 μM inhibits HSV-1 infection of Vero cells by 50%, i.e., the EC₅₀ dose. Thus, the data in Examples 1 and 2 show that the EC₅₀ dose is about 6.4-fold lower than the CC₅₀ dose.

### Example 3

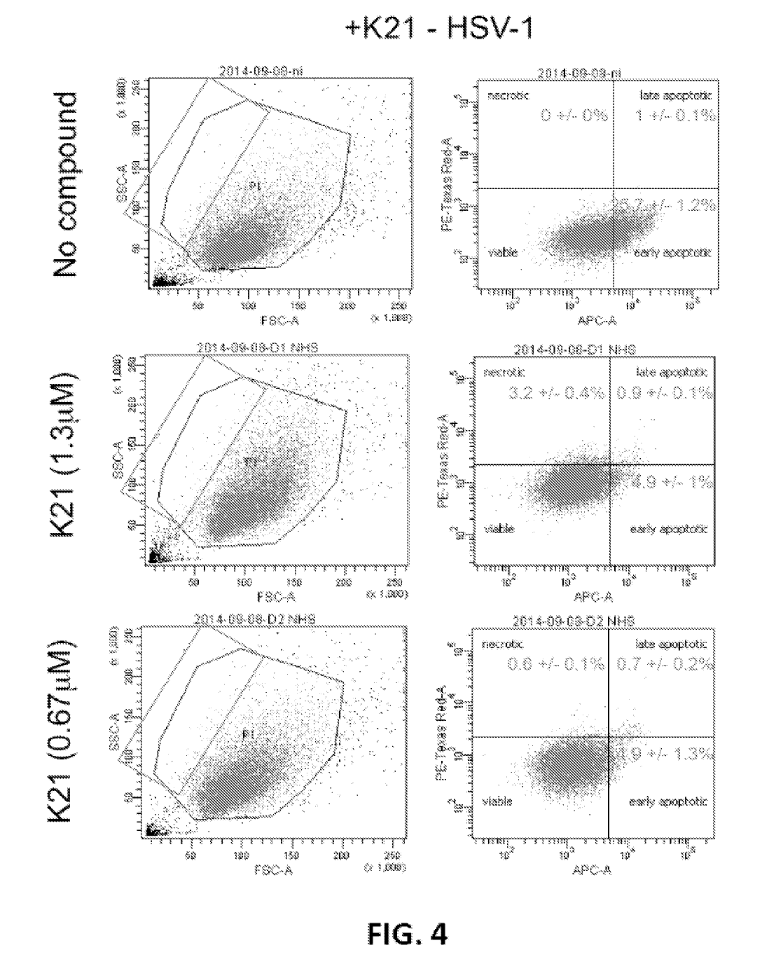

The effect of K-21 on induced cytopathy in uninfected Vero cells was determined using cell flow cytometry of cells following exposure to compound K-21, and the data are shown in FIG. 4. The data are shown in FIG. 4, and the non-viable cell populations determined by this method are indicated within the boxed areas shown. The graphs on the left side of the page show the correlation of side-scattered light (SSC) with forward-scattered light (FSC), and the boxed areas show the regions wherein the non-viable cell population is found. The graphs on the right side the correlation of allophycocyanin (indicated as APC on the x-axis) and Texas Red (y-axis). Also shown for each graph on the right are the quadrants wherein necrotic, late apoptotic, viable, and early apoptotic cell populations are found. The concentration of K-21 that the Vero cells were exposed to is shown to the left of each row of graphs.

### Example 4

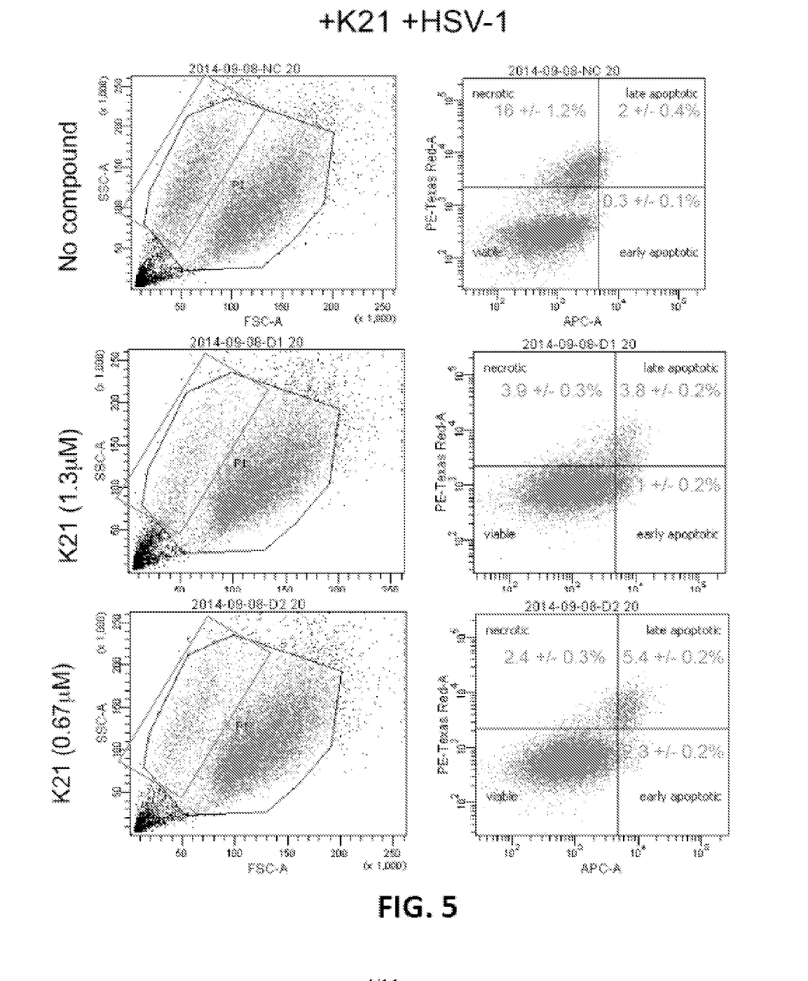

The effect of K-21 on induced cytopathy in infected Vero cells was determined using cell flow cytometry of cells following exposure to compound K-21, and the data are shown in FIG. 5. The data are shown in FIG. 5, and the non-viable cell populations determined by this method are indicated within the boxed areas shown. The graphs on the left side of the page show the correlation of side-scattered light (SSC) with forward-scattered light (FSC), and the boxed areas show the regions wherein the non-viable cell population is found. The graphs on the right side the correlation of allophycocyanin (indicated as APC on the x-axis) and Texas Red (y-axis). Also shown for each graph on the right are the quadrants wherein necrotic, late apoptotic, viable, and early apoptotic cell populations are found. The concentration of K-21 that the Vero cells were exposed to is shown to the left of each row of graphs. Thus, the data in Examples 3 and 4 further show that the cytopathic effect of K-21 is specific to infected cells.

### Example 5

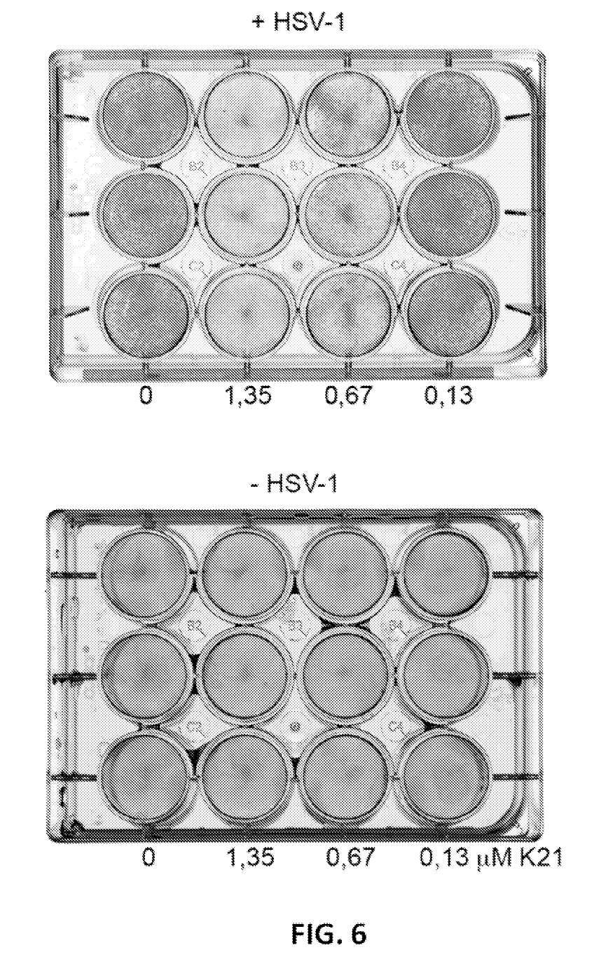

The induction of cell death in uninfected (indicated as “−HSV-1” in FIG. 6) and infected (indicated as “+HSV-1” in FIG. 6) Vero cells treated with compound K-21 was determined and the data are shown in FIG. 6. The experiment was conducted at 72 hr post-infection or sham-infection for infected and uninfected cells, respectively. The assay is a standard plaque assay carried out under standard conditions. The data show tthat K-21 induces cell death in the infected Vero cells, but not in the uninfected cells, at the concentrations of K-21 tested.

### Example 6

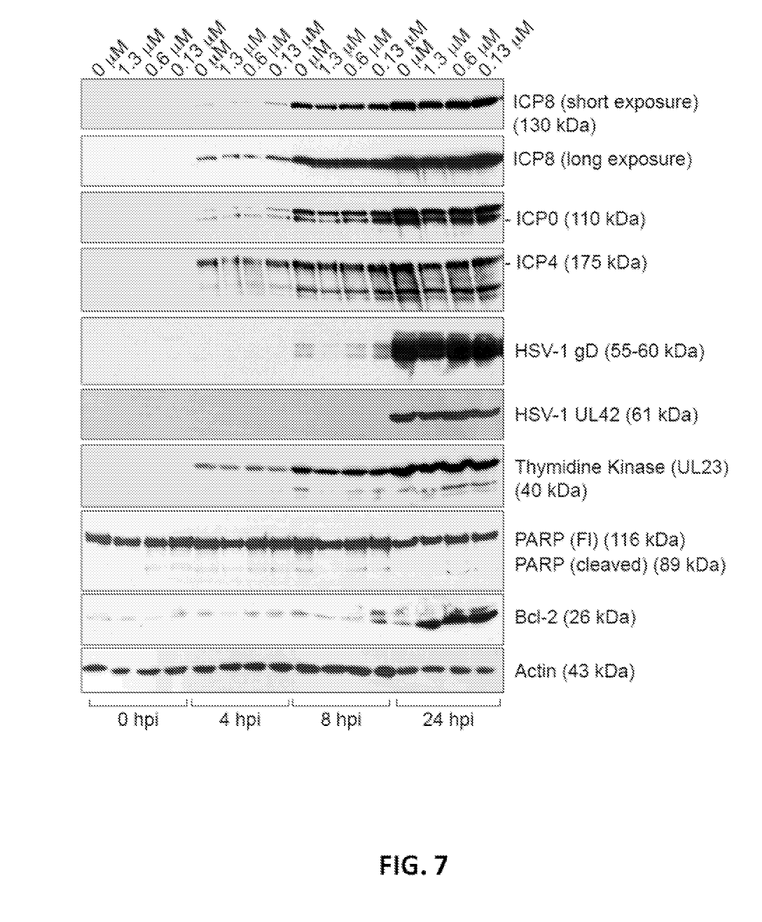

The expression of the certain proteins in Vero cells following infection by HSV-1 was determined by Western blot analysis, and the data are shown in FIG. 7. The concentrations of compound that cells were exposed to foor the various sample lanes are indicated at the top of the figure, the time post-infection that the sample was obtained is shown at the bottom of the figure, and the identity of the protein determined in the blot panel is shown on the right side of the figure. The data show that compound K-21 downregulates ICP0, ICP4 and ICP8 expression in Vero cells infected with HSV-1. However, UL42 (DNA polymerase subunit), as well as viral infectivity associated thymidine kinase, gene expression are not altered in infected cells exposed to compound K-21. Moreover, the data show that only HSV-1 infected cells induce Bcl-2 expression, beginning after about 8 hrs of viral infection.

These data, taken together with the other data in the Examples, indicates that compound K-21 possesses anti-HSV-1 activity and that it down-regulates HSV-1 induced cell death. Without wishing to be bound by a particular theory, it is possible that the down-regulation by compound K-21 of HSV-1 induced cell death is via an upregulation by compound K-21 of Bcl-2 protein expression. Bcl-2 is a known anti-apoptotic protein. The data are consistent with K-21 having activity that specifically disrupts the integrity of infected cells. Without wishing to be bound by a particular theory, it is believed that the K-21 has membrane rupturing potential associated with one or more aspects of molecular structure.

### Example 7

Nude mice were injected with human head and neck cancer cell line (HTB41/-43). After the tumors were established in the abdomen, some tumors were injected with PBS (sham), some with Cisplatin, and some with 5% by weight K-21. The size of the sham injected tumors grew most rapidly over 10 days. The Cisplatin injected animal tumors grew the least, while the 5% K-21 injected tumors grew slower than the shams but more rapidly than the Cisplatin injected animals. 5% K-21 appears to have anti-tumor activity.

### Example 8

A hand sanitizer (sold under the brand name of fiteBac) containing 5% by weight of K-21 in a hydrophobic base was evaluated for its anti-microbial and anti-viral activity using a new laboratory model (Rapid Agar Plate Assay). Sterile agar places were pretreated with fiteBac hand sanitizer, an ethanol-based hand sanitizer, or liquid soap. Then the plates were inoculated with S. aureus or E. coli. For anti-viral activity, mammalian cell lines were grown to confluency and infected with noroviruses (murine norovirus or feline calcivirus). The number of dead cells was quantitated. Liquid soap had no effect on bacteria or viruses. Both the ethanol-based hand sanitizer and fiteBac hand sanitizer were very effective at killing S. aureus but not E. coli. Both hand sanitizers killed more noroviruses than bacteria. 5% K-21 is a very effective anti-viral agent.

### Example 9

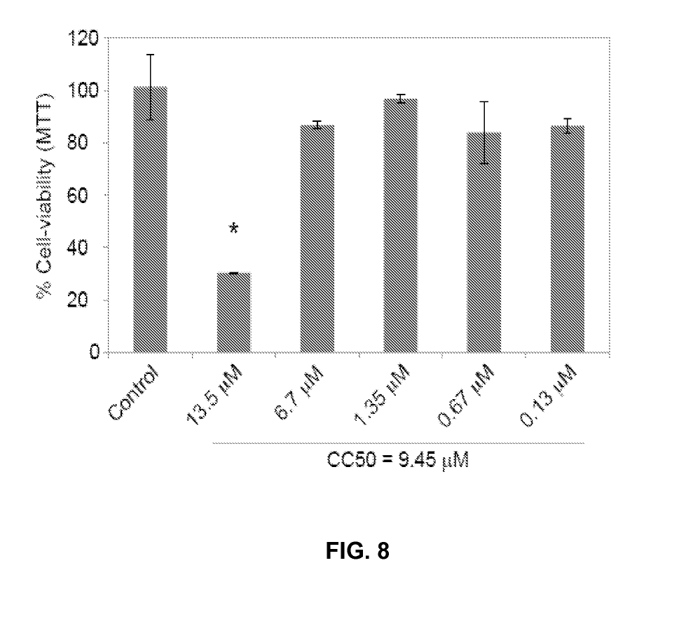

A cell viability (MTT) assay in primary human foreskin fibroblasts (HFFs) was used to determine the cytotoxic dose (CC50) of K-21. HFFs were seeded into 96-well plates (10³ cells/well). At 24 hours in culture with varying amounts of K-21, the viability was determined relative to solvent control-treated cells (Control) from two repeated experiments performed in triplicate. *p<0.05. CC50 value was calculated to be 9.45 μM from the calculated slope equation. The results are shown in FIG. 8.

### Example 10

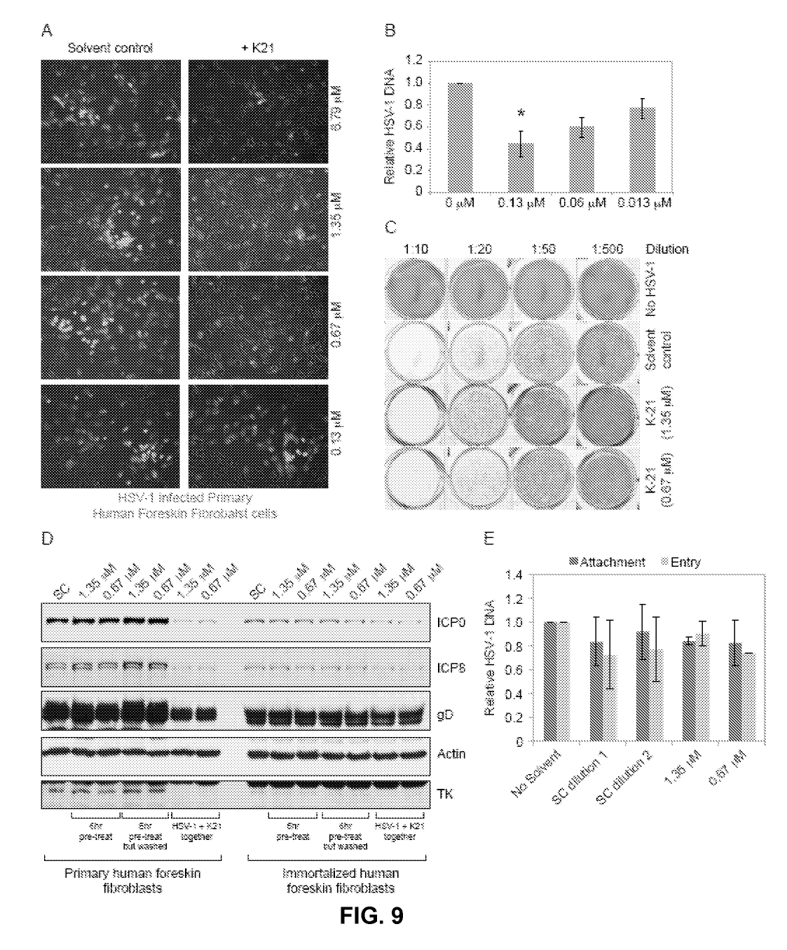

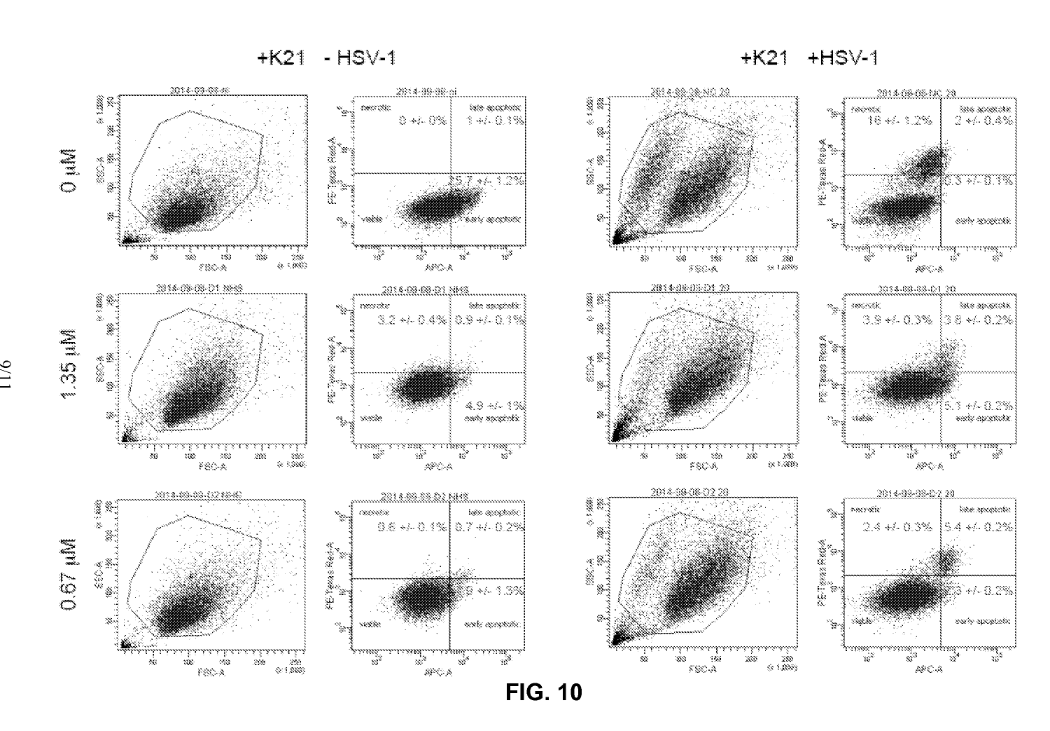

K-21 inhibits HSV-1 infection in primary HFFs, as shown in FIG. 9.

In FIG. 9(A): HFFs were infected with HSV-1 that express eGFP in infected cells. Infected cells were treated with either solvent control or K-21 at different concentrations. At 24 hours, post infection, cells were imaged using an epifluorescence microscope.

In FIG. 9(B): Total genomic DNA was extracted from a parallel set of experiments and HSV-1 DNA amount in the infected cells was quantified by qPCR.

In FIG. 9(C): In a similar set of experiments, infected cells were lysed after 24 hours of infection and cell lysates were transferred to freshly seeded Vero cells at different dilutions. Plaque assays were carried to check the infectious progeny.

In FIG. 9(D): Primary HFFs as well as transformed HFFs were infected with HSV-1 for 24 hours in presence or absence of K-21 (with solvent control—SC). One set of cells were pre-treated with K-21 for 6 hours and HSV-1 was added to the cells after 6 hours. In another set, cells were treated with K-21 for 6 hours and then washed thoroughly with PBS and then infected with HSV-1. In a third set of experiments, both HSV-1 and K-21 were added to the cells at the same time. Total lysate from 24 hour infected cells were used for immunoblotting.

In FIG. 9(E): The effect of K-21 on HSV-1 entry and attachment was studied by qPCR.

### Example 11

K-21 inhibits HSV-1 infection-induced cytopathic effects, as shown in FIG. 10. Vero cells were infected with HSV-1 in the presence of K-21 at different dilutions or were grown in the absence of HSV-1 infection but with the same concentrations of K-21. 24 hours post infection, cells were processed for flow cytometry to quantify number of apoptotic or necrotic cells. Data represents one of the triplicate experiments.

### Example 12

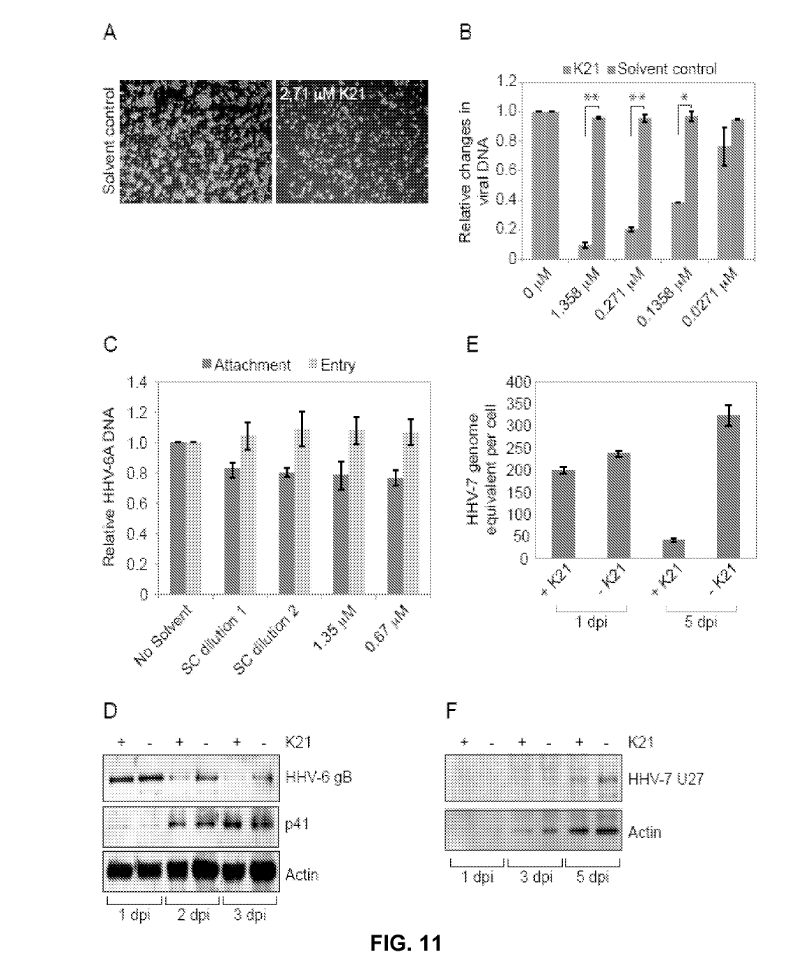

K-21 inhibits HHV-6 and HHV-7 infection, as shown in FIG. 11.

In FIG. 11(A): HSB-2 cells were infected with HHV-6A that expresses mCheery protein in infected cells. Infected cells were treated either with solvent control or K-21. At 72 hours post infection, cells were imaged using an epifluorescent microscope.

In FIG. 11(B): Total genomic DNA was extracted from a parallel set of experiments and HHV-6 DNA amount in infected cells was quantified by qPCR.

In FIG. 11(C): Effect of K-21 on HHV-6A entry and attachment were studed by qPCR, where SC=solvent control.

In FIG. 11(D): Effect of K-21 on HHV-6 replication and growth were studied by immunoblotting. HHV-6A infected HSB-2 cells were mixed with uninfected HSB-2 cells at a ratio of 1:5 and were kept either in the presence of K-21 or solvent control for different time intervals. Total protein lysates were prepared and were analyzed for expression of HHV-6 early protein p41 (marker for viral replication) and late protein gB (marker for final viral particle formation).

In FIG. 11(E): Total genomic DNA in infected cells were quantified by qPCR for HHV-7.

In FIG. 11(F): Effect of K-21 on HHV-7 replication and growth were studied by immunoblotting. HHV-7 infected SupT-1 cells were mixed with uninfected SupT-1 cells at a ratio of 1:10 and were kept either in the presence of K-21 or solvent control for different time intervals. Total protein lysates were prepared and were analyzed for expression of HHV-7 late protein U27. Actin was used as loading control.

### Example 13

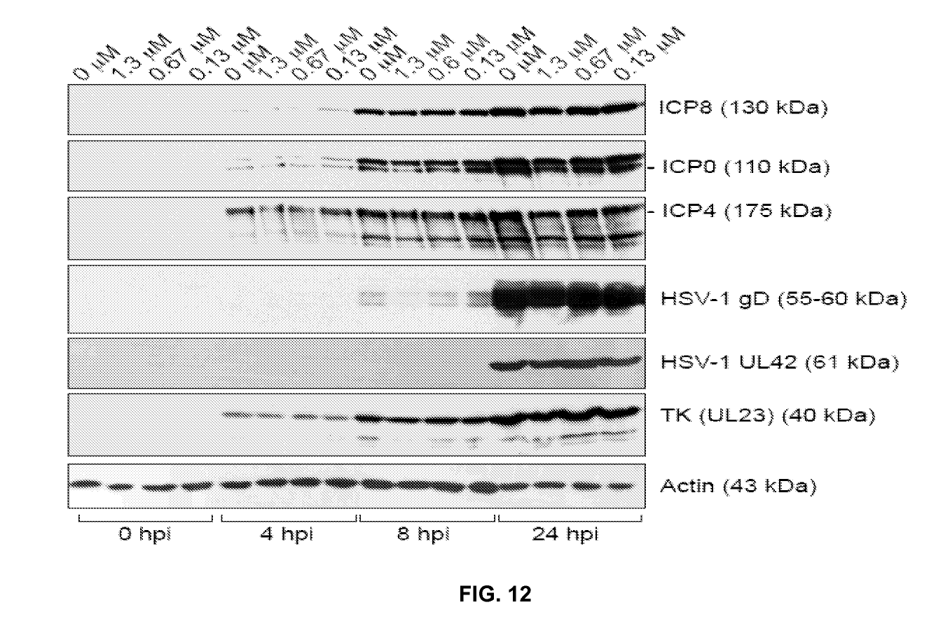

K-21 inhibits HSV-1 infection in Vero cells, as shown in FIG. 12. The effect of K-21 on HSV-1 replication and growth were studied by immunoblotting. Vero cells were infected with HSV-1 at a MOI of 5. Total protein lysates were prepared at different time intervals and were analyzed for expression of different HSV-1 proteins. Action was used as a loading control.

### Example 14

Chronic wounds in diabetics are difficult to heal because the blood supply to the wound is compromised. Using diabetic (db/db) mice (10 per group), 6 mm diameter punch biopsy wounds were created on the shaved backs of mice. These wounds were then inoculated with Pseudomonas aeruginosa (PA01) biofilms two days post-wounding and were then covered by unmediated bandages or bandages coated with 5% K-21, an organosilicone-containing one mole of quaternary ammonium compound and 3 moles of methacrylate moieties. After 2 weeks, the animals were sacrificed and the wounds were excised for histologic and flow cytometry and processed for inflammatory indices, histology, tissue necrosis and epidermal hyperplasia in untreated and 5% K-21 treated wounds.

### Example 15

K-21 was dissolved in acetone to enhance its ability to wet the shaved skin on the backs of experimental mice. Mice in the positive control group were treated with a topical chemical carcinogenic agent, dimethybena(α)anthracene (DMBA), dissolved in acetone. This group was included to demonstrate that known chemical carcinogens dissolved in acetone and topically applied to shaved skin of mice can induce tumors with a single topical application, thereby validating the animal model.

Thirty adult C57/BL6 mice were randomly divided into three groups of ten animals each to evaluate the topical irritation and potential anti-inflammatory properties of K-21. The hair on the back of all mice were shaved off with electric clippers.

Group 1 animals received topical application of K-21 (100 μg/100 μL) in acetone twice a week for two weeks (days 3, 6, 9, and 12) over the shaved area. These animals received no other treatment. Group II mice received a topical single dose of DMBA (100 μg/100 μL) in acetone over the shaved skin on the animals back on day 7. Group III animals received the same treatment as Group II mice on day 7 plus they were treated with topical K-21, twice a week before (days 3, 6) and one week after (days 9 and 12) a single DMBA application on day 7.

All animals were weighed daily, before and during the study to compare the weight gains of animals in all three groups. The behavior (eating, drinking, etc.) was monitored on a daily basis to determine if there was any gross skin irritation or initiation of tumors. All animals were sacrificed at he end of the second week using and i.p. overdone of pentobarbital.

Flow cytometry analyses: The skin and some associated soft tissues on the backs of all mice were excised. Representative portions of the skin and dermis were prepared for flow cytometry analysis by being divided into single cell suspensins using a 100 μm cell strainer followed by centrifugation (1500 rpm, 10 mm). Analysis of cells for detection of the inflammatory cytokine IL-1 was performed by fixing, permeabilizing, and staining of cells and running flow cyctopmetry using a FACS Calibur BD Flow Cytometer.

Morphological analysis: Other representative portions of treated skin were fixed and processed for light microscopy. Sections were stained with H&E in paraffin embedded tissue from the affected areas, as previously described (Baban et al., Physiologic control of IDP competence in splenic dendritic cells. J. Immunol. 2011: 187(5):2320-35).

Results: All animals survived the two-week experiment. There were no difference in food or water intake or weight gain among the three groups of mice.

No tumors or signs of epithelial or dermal swelling or inflammation were seen in any of the Group I mice (application of K-21 alone). Tumors formed in all ten of the group II mice that received a single dose of DMBA. The tumors were about 0.5 cm in diameter at day 14. Tumors formed in all ten animals in group III mice that had received topical K-21 application before and after a single topical application of DMBA.

Micrographs show tumors growing on the back of a mouse topically treated with one application of DMBA on day 7 (group II animal) that was sacrificed at day 14. Multiple tumors about 0.5 cm in diameter were seen on the shaved back of the animal. No tumor growth was seen macroscopically on the group I animal (K-21 alone).

An H&E stained section showed keratinized epithelium beneath which were multiple dermal harir follicles surrounded by excessive fibrous tissue infiltrated with round cells (inflammation) in a skin section taken from a tumor seen in the skin of a group II animal. An H&E stained section of a group I mouse showed normal thickness. There was little fibrous tissue around dermal hair follicles and no histological evidence of dermal inflammation or microscopic cancer cells. A macroscopic view of a representative mouse from group III mice showed a similar tumor size to group II mice.

Flow Cytometry: Most cells of Group I mice were negative for IL-1. In negative controls, only 0.05% of the total number of cells were IL-1 positive. For group II, 6% of the total number of cells were IL-1 positive. For group III, 1% of the total number of cells were IL-1 positive (p<0.05), showing a significant reduction in IL-1 expression compared to group II tumor cells.

The results of this study indicate that four topical applications of K-21 to the group animals produced no gross or microscopic evidence of skin irritation nor abnormalities. In group II animals, all mice exhibited tumor formation by day 14 (when sacrificed). In addition to tumor cells formation, the dermal tissues exhibited inflammatory cells. This is apparently due to cancer cells releasing molecular mediators, such as the pro-inflammatory cytokine, IL-1.

The lower levels of IL-1 positive tumor cells in mice in group II may have been due to the barrier properties of residual organosilanes left on the skin from K-21 topical treatments prior to the DMBA application. Arguing against this ideas is the fact that group III animals had the same number and size of tumors as grou II animals that only had received DBMA without K-21. However, it is belieed that the residual K-21 on the skin was carried down hari follicels during topical application of DMBA in acetone. The cationic quaternary ammonium chloride in K-21 may have inhibited MMPs released by inflammatory cells. Preliminary testing of K-21 on rh cathepsin K activity in vitro revealed strong inhibition.

When ranges are used herein for physical properties, such as molecular weight, or chemical properties, such as chemical formulae, all combinations, and subcombinations of ranges specific embodiments therein are intended to be included.

The disclosures of each patent, patent application, and publication cited or described in this document are hereby incorporated herein by reference, in their entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A pharmaceutical composition, comprising at least one compound of formula I: 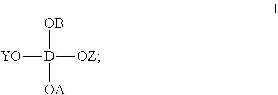
wherein:
D is independently Si, Ti, Al, or Zr;
A, B, Y, and Z are each independently selected from the group consisting of H, (C₁-C₈)alkyl, trifluoro-substituted (C₁-C₈)alkyl, and 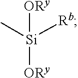
R^{b} is independently 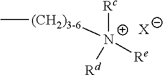
wherein:
R^{c} is (C₁-C₂)alkyl;
R^{d} is (C₁-C₂)alkyl or phenyl;
R^{e} is (C₆-C₂₂)alkyl;
X⁻ is an anion selected from the group consisting of chloride, bromide, fluoride, iodide, sulfonate, and acetate;
each R^{y} is, independently, H, (C₁-C₈)alkyl, or trifluoro-substituted (C₁-C₈)alkyl; and
wherein at least one of A, B, Y, and Z is 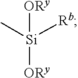
and
a pharmaceutically-acceptable excipient.

2. The pharmaceutical composition of claim 1, wherein each of A, B, Y and Z is

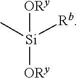

3. The pharmaceutical composition of claim 1, wherein D is Si.

4. The pharmaceutical composition of claim 1, wherein R^{c} is (C₁)alkyl.

5. The pharmaceutical composition of claim 1, wherein R^{d} is (C₁)alkyl.

6. The pharmaceutical composition of claim 1, wherein R^{e} is (C₁₈)alkyl.

7. The pharmaceutical composition of claim 1, wherein X is chloride.

8. The pharmaceutical composition of claim 1 wherein R^{b} is independently —(C₃-C₆ alkylenyl)—(dimethyl)—(C₆-C₂₂ alkyl) quaternary ammonium chloride or —(C₃-C₆ alkylenyl)—(methyl)—(phenyl)—(C₆-C₂₂ alkyl) quaternary ammonium chloride.

9. The pharmaceutical composition of claim 8, wherein R^{b} is —(C₃ alkylenyl)—(dimethyl)—(C₁₈ alkyl) quaternary ammonium chloride.

10. The pharmaceutical composition of claim 1, wherein each R^{y} is H.

11. The pharmaceutical composition of claim 1, wherein the compound of formula I has the formula:

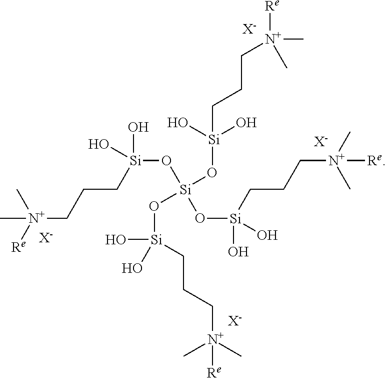

12. The pharmaceutical composition of claim 1, wherein the compound of formula I has the formula:

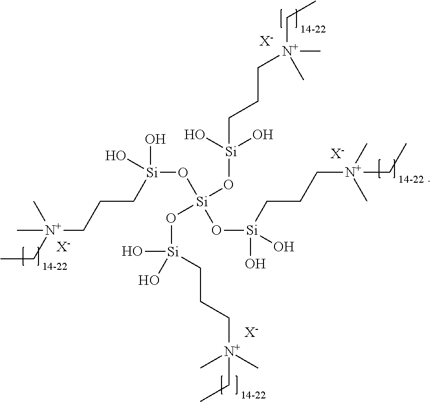

13. The pharmaceutical composition of claim 1, wherein the compound of formula I has the formula:

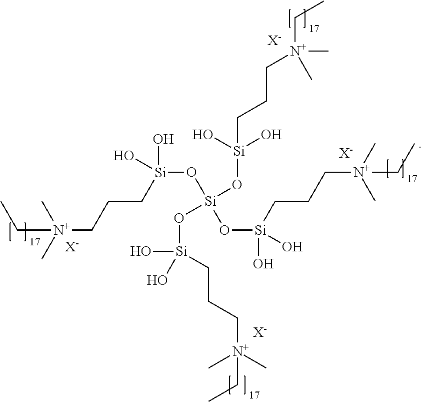

14. The pharmaceutical composition of claim 11, wherein X⁻ is chloride.

15. The pharmaceutical composition of claim 1, further comprising at least one therapeutic agent.

16. The pharmaceutical composition of claim 15, wherein the therapeutic agent comprises at least one agent selected from an antiviral agent, an antibacterial agent, an antituberculosis agent, an antifungal agent, and combinations thereof.

17-30. (canceled)

31. The pharmaceutical composition of claim 1, further comprising ethanol.

32. A method of treating an infection in a subject, comprising administering to the subject an effective amount of the pharmaceutical composition of claim 1.

33-66. (canceled)

67. A method of reducing pain associated with or hastening healing of a cutaneous or mucosal membrane lesion caused by a herpes viral infection in a mammal, comprising administering to the mammal an effective amount of the pharmaceutical composition of claim 1.

68-70. (canceled)

71. A kit comprising the pharmaceutical composition of claim 1, and at least one of:
a) at least one therapeutic agent known to treat a viral infection;
b) at least one therapeutic agent known to treat a bacterial infection;
c) instructions for treating a viral infection;
d) instructions for treating a bacterial infection;
e) instructions for administering the pharmaceutical composition in connection with treating a viral infection; and
f) instructions for administering the pharmaceutical composition in connection with treating a bacterial infection.

72-75. (canceled)
